(19) United States
(12) Patent Application Publication
(10) Pub. No.: US 2012/0082978 A1
(43) Pub. Date: Apr. 5, 2012

(54) Cell Analysis On Microfluidic Chips

(76) Inventors: Linda Pilarski, Alberta (CA); Carina Debes-Marun, Alberta (CA); Patrick Pilarski, Alberta (CA); Christopher Backhouse, Alberta (CA); Vincent Sieben, Alberta (CA); Govind Kaigala, Alberta (CA)

(21) Appl. No.: 12/310,900

(22) PCT Filed: Sep. 17, 2007
(86) PCT No.: PCT/CA2007/001641

§ 371 Date: Sep. 12, 2011

Related U.S. Application Data
(60) Provisional application No. 60/844,643, filed on Sep. 15, 2006.

(51) Int. Cl. C12Q 1/68; G06K 9/00; C12M 1/34

(52) U.S. Cl. 435/6.11; 435/287.2; 382/137

(57) ABSTRACT

The present invention provides for a method of implementing fluorescent in situ hybridization (FISH) or other cellular analysis processes using intact cells within a microfluidic, chip-based, apparatus. The invention further provides for a method of cellular immobilization within a microfluidic device. Also provided is a method for automated analysis of FISH or other cellular analysis using discrete colormetric probes.

### RELATED APPLICATION

This application claims the benefit of U.S. Provisional Application Ser. No. 60/844,643, filed Sep. 15, 2006, filed under 35 U.S.C. 119(e). The entire disclosure of the prior applications are hereby incorporated by reference.

### FIELD OF THE INVENTION

The present invention pertains to the field of cellular analysis.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

Fluorescent In Situ Hybridization (FISH) is a safe, stable clinical test for abnormal genetic mutations in human cells. One particular use of FISH is the detection of anomalous chromosome structures in patients with cancers of the blood and immune systems (hematopoietic disorders such as multiple myeloma) (Fluorescence In-Situ Hybridization: A Practical Approach, Beatty, B. et al., Oxford University Press (2002)). It has been shown that certain mutations can have a dramatic effect on a patient's response to treatment and overall survival (Gertz, M. A. et al., Blood, 106(8):2837-2840 (2005); Dewald, G. W. et al., Blood, 106(10):3553-3558 (2005); Jaksic, W. et al., J. Clin. Onc., 23(28):7069-7073 (2005)). Through the use of FISH, it is possible to determine the predicted response of a given patient and assign treatments accordingly.

One drawback of the current FISH analysis protocols is that a human observer is still required to manually assess the outcome of the tests (Theodosiou, Z. et al., Cytometry A, 71A:439-450 (2007); Lerner, B. et al., Ieee-Acm Transactions On Computational Biology and Bioinformatics, 4:204-215 (2007)). This involves examining individual cells in populations exceeding several hundred to thousands of cells. As 100 to 200 observable cells are needed to accurately assess patient status, it is evident that an automated system is needed to help laboratory personnel provide rapid diagnostic verdicts. While computer systems have been designed to address these difficulties, most only perform well on noise-free precision-focused images from expensive optical setups. None have been fully integrated with any form of miniaturized diagnostic device, and none have been validated to a convincing degree through clinical trials (Theodosiou, Z. et al., Cytometry A, 71A:439-450 (2007); Lerner, B. et al., Ieee-Acm Transactions On Computational Biology and Bioinformatics, 4:204-215 (2007)). Human intervention is still required in analysis systems (Theodosiou, Z. et al., Cytometry A, 71A:439-450 (2007)).

FISH is a safe and efficient way to test for the number and structure of chromosomes in the nucleus of human cells. By attaching fluorescent-labeled pieces of complementary DNA to sections of chromosome it is possible to visually identify the relative arrangement and location of specific genetic sequences. FISH quickly replaced radiation-based detection methods as a way to identify genetic mutation, as it could be safely done in a laboratory setting (FISH: a practical approach, Oxford Press (2002)). This is important, as deviations in the number and layout of chromosomes are clinically important characteristics of solid tumors and hematopoietic cancers such as multiple myeloma. Efficient identification of selected genetic mutations can allow doctors to tailor the type and amount of treatment to best suit the biological receptivity of the patient (Gertz, M. A. et al., Blood, 106(8):2837-2840 (2005)).

Interphase FISH is especially important in the analysis of hematopoietic cancers, for example multiple myeloma patient samples. A large proportion of patients with multiple myeloma will have mutations of their chromosome sets—for instance, sections of chromosome may be swapped or out of place. This is called a translocation. With FISH labeling, it is possible to detect the number of cells baring translocations among the whole cellular population in a patient.

In general there are four major stages to FISH analysis. The first stage is probe preparation. FISH probes are labeled with a specific fluorophore and consist of specially designed sections of nucleic acids that are complementary to a narrow sequence of chromosomal nucleic acids (Fluorescence In-Situ Hybridization: A Practical Approach, Beatty, B. et al., Oxford University Press (2002)). Pre-designed probes can be purchased prior to experimentation to match the nucleic acids under examination and the optic constraints of the imaging system. In the second step, both the probe and the sample of nucleic acids (typically still inside the nucleus of a whole cell in an interphase FISH assay) are denatured resulting in a multiplicity of single stranded code sections. In the third step, the probe is allowed to hybridize, or find the complementary nucleotide pairs on the denatured DNA strands. This process is graphically presented in FIG. 1.

After hybridization, the whole cell is analyzed using fluorescent microscopy, and a series of images are captured—one color channel for each probe fluorescent response wavelength. It is important to note that the sample image channels contemplated by the present invention have the color artificially injected into them after capture; the imaging system simply records intensity maps through a series of optical filters and allows the user to best assign color information to aid in visual analysis. One can then view the location of the hybridized probes by identifying the areas of the image with high levels of fluorescence in a particular probe frequency range (Fluorescence In-Situ Hybridization: A Practical Approach, Beatty, B. et al., Oxford University Press (2002)). In clinical use this process is carried out on many cells in parallel, usually with the sample population immobilized on a set of microscope slides and surrounded by a probe solution.

Once the physical arrangement of the probes can be visualized, it is possible to determine the presence of anomalous translocations, wherein sections of chromosomal DNA have changed location relative to each other, compared to that observed in healthy cells. Among the many ways this can be done are, for example, through the use of two probe types: dual-fusion probes and break-apart probes. With dual fusion probes, the two chromosomes under analysis (for example chromosomes 4 and 14 in multiple myeloma) will each be tagged with a unique probe color. If the translocation is present, this will result in two different coloured probes in close proximity (“close” is defined as within a probe diameter or less of each other). This can be seen in FIG. 2. In the alternative, “break apart” probes can be used wherein the coloured probes are normally in close proximity (“close” is defined as within a probe diameter or less of each other), and if there is a translocation event, the two probes will be observed to no longer be in close proximity.

Although human chromosomes have been studied for over a century, it was the introduction of FISH analysis techniques, particularly interphase FISH, in the mid-1980s that allowed researchers to rapidly investigate and understand the chromosomal basis of many genetic diseases and cancers (Nath, J. et al., Biotech Histochem., 75:54-78 (2000); Swiger, R. R. et al., Environ. Mol. Mutagen, 27:245-254 (1996); Fluorescence In-Situ Hybridization: A Practical Approach, Beatty, B. et al., Oxford University Press (2002); Introduction to Fluorescence In Situ Hybridization: Principles and Clinical Applications, Andreeff, M., New York: Wiley-Liss (1999)). Interphase FISH is more sensitive than conventional cytogenetic methods for detecting chromosomal changes, for example, the translocation t(4;14)(p16;q32) found in multiple myeloma (MM) patients which is not detectable by cytogenetic methods. Since some changes are not easily found by conventional methods, and are readily detectable by interphase FISH this technique has become an indispensable tool for gene mapping and characterization of chromosome aberrations (Tonnies, H., Trends in Molecular Medicine, 8:246-250 (2002); King, W. et al., Mol. Diagn., 5:309-319 (2000); Gertz, M. A. et al., Blood, 106:2837-2840 (2005)). This named translocation, along with other abnormalities have been associated with lower survival rates, and patients harbouring these abnormalities, do not respond well to conventional or high dose treatments (Gertz, M. A. et al., Blood, 106:2837-2840 (2005); Dewald, G. W. et al., Blood, 106:3553-3558 (2005); Jaksic, W. et al., J. Clin. Oncol., 23:7069-7073 (2005)). Since some of the therapies have secondary effects that greatly compromise quality of life, it is necessary to determine the appropriate therapeutic approach for each patient. Consequently, interphase FISH should be employed in a clinical setting to recognize, for instance t(4;14)(p16.3;q32), allowing clinicians to make highly informed decisions regarding patient treatment. However, the complexity and numerous protocol steps involved in a typical interphase FISH analysis are labour intensive and time consuming, taking days to complete. In particular, the cell preparation and probe hybridization portions of the experiment take approximately 80% of the overall time. Furthermore, the probes required to perform FISH are relatively expensive (approximately $90 per test), and a highly trained specialist is required to interpret the staining patterns. Together, these factors have prevented FISH from becoming a commonly employed screening technique. The art is in need of a method, apparatus and system capable of reducing the labour, time and cost in FISH, to the extent that the more widespread application of microchip-based FISH can be expected in the future.

The process of integrating and miniaturizing conventional techniques onto microfluidic platforms is widely referred to as the creation of Micro-Total Analysis Systems (μTAS). It has been demonstrated that these are potentially superior platforms for biological assays when compared with many conventional analytical tools (Dittrich, P. S. et al., Anal. Chem., 78:3887-3907 (2006); Manz, A. et al., Sensors and Actuators B-Chemical, 1:244-248 (1990)). In μTAS, planar microchips incorporate a network of embedded microchannels that transport the sample from one manipulation to the next, enabling both precise control of reagents and automation of several consecutive steps (Manz, A. et al., Sensors and Actuators B-Chemical, 1:244-248 (1990) Lichtenberg, J. et al, Talanta, 56:233-266 (2002)), while leading to a significant reduction in total analysis time. For instance, hybridization is the most time-intensive part of DNA microarray technologies and there are considerable research efforts aimed at improving the speed and efficiency of DNA hybridization (Heller, M. J., Ann. Rev. Biomed. Eng., 4:129-153 (2002)). In traditional microarray hybridization approaches, the reaction rate is in part limited by molecular diffusion; therefore, it takes a significant amount of time for the target to find and hybridize to its complementary probe (Kamholz, A. E. et al., Biophys. J., 80:155-160 (2001); Hatch, A. et al., Nat. Biotechnol., 19:461-465 (2001); Kamholz, A. E. et al., Anal. Chem., 71:5340-5347 (1999); Kamholz, A. E. et al., Sensors and Actuators B-Chemical, 82:117-121 (2002)). To overcome this diffusion transport limitation, several groups have implemented electrokinetic or mechanical mixing of probes and targets on microchips (Erickson, D., et al., Anal. Chem., 76:7269-7277 (2004); Smith, D. E. et al., Macromolecules, 29:1372-1373 (1996); Sorlie, S. S., Macromolecules, 23:487-497 (1990); Kim, J. H. S. et al., Sensors and Actuators B-Chemical, 113:281-289 (2006); Vanderhoeven, J. et al., Electrophoresis, 26:3773-3779 (2005)). The agitation introduced by these approaches results in a 2- to 20-fold reduction in hybridization/analysis time.

When an electric field is applied during hybridization, mobile DNA targets can be precisely controlled, thereby allowing continual replenishment or recirculation of targets to the immobile probes on the channel surface (Erickson, D. et al., Anal. Chem., 76:7269-7277 (2004); Santiago, J. G. et al., Anal. Chem., 73:2353-2365 (2001); Oddy, M. H. et al., Anal. Chem., 73:5822-5832 (2001); Biddiss, E., Anal. Chem., 76:3208-3213 (2004)). In a recent example, Erickson et al. improved upon DNA microarray techniques by implementing an H-type channel fabricated on a glass and PDMS microfluidic chip that permitted electrokinetic delivery of targets (Erickson, D. et al., Anal. Chem., 76:7269-7277 (2004)). By restricting the channel height to 8 μm, they reduced the time it takes for a DNA target to vertically diffuse from the top of the channel to the bottom where the complementary probes are located and hybridization can occur. When physical confinement is combined with a continual delivery of fresh targets by electrokinetic transport, the hybridization time is reduced 20-fold. Equally important advantages include smaller volumes of sample and reagent usage, portability, and high density parallel processing.

Alternatively, it has been demonstrated with DNA microarrays that volumetric flow can also be utilized to decrease the hybridization reaction time. Kim (Kim, J. H. S. et al., Sensors and Actuators B-Chemical, 113:281-289 (2006)) and Cheek (Cheek, B. J. et al., Anal. Chem., 73:5777-5783 (2001)) determined that a continual flow of targets at the highest volumetric flow rate and the lowest channel height yielded the fastest and most efficient hybridization. Indeed, the concept is similar to electrokinetic pumping, employing a low channel height to minimize the vertical diffusion distance and a volumetric flow that provides a constant source of fresh DNA probes. Recently, mechanical pumps and valves have been incorporated within microfluidic chips, providing a high level of integrated fluid control (Stone, H. A. et al., Annual Review of Fluid Mechanics, 36:381-411 (2004); Skelley, A. M. et al., Proc. Natl. Acad. Sci. USA, 102:1041-1046 (2005); Quake, S. R. et al., Science, 290:1536-1540 (2000)). One of the key benefits of these integrated and miniaturized valves and pumps is that they have lower dead volumes and therefore waste less of the expensive reagents.

Since conventional interphase FISH techniques are dependent on diffusion-limited hybridization, there is potential for hybridization enhancements. Yet, in interphase FISH the samples are immobilized as whole cells and chromosomes, as opposed to the short DNA fragments used on DNA microarrays. Unlike DNA microarrays, the hybridization process within a cell is substantially more complicated because the probes, which are on the order of kilobase pairs in length, must first diffuse to the cell wall, traverse it, and then find their specific binding site within three billion base pairs of chromosomal DNA. With DNA microarray technology, it has been shown that as the number of hybridization sites is increased (each site with a different sequence), a competitive process between the various different fragments significantly lengthens the time taken for specific hybridization (Peterson, A. W. et al., Nucleic Acids Res., 29:5163-5168 (2001); Erickson, D. et al., Anal. Biochem., 317:186-200 (2003)). In the case of interphase FISH, by the same mechanism, the large range of distinct potential binding sites within a cell (orders of magnitude more dense than in DNA microarrays) may be expected to increase the time taken for hybridization. Moreover, when targeting chromosomes with interphase FISH, the hybridization must occur within the physical volume of a cell nucleus and within packed chromatin (Introduction to Fluorescence In Situ Hybridization: Principles and Clinical Applications, Andreeff, M., New York: Wiley-Liss (1999)). The diffusion is therefore hindered by the presence of RNA, enzymes, and various proteins, such as histones that bond to DNA. Clearly then, although interphase FISH is in some degree dependent upon slow diffusion mechanisms, the process of hybridization is far more complex than in DNA microarray work. Nevertheless, performing interphase FISH in the physical confinement of a microchannel permits precise control of the hybridization kinetics and enables optimal reagent usage, leading to a reduction in cost and hybridization time.

### SUMMARY OF THE INVENTION

The present invention also provides for an automated computer vision system capable of assessing the presence, absence and location of a luminescent probe within a cell or population of cells comprising a computer readable memory a computer and an optical imaging device all in digital communication with each other wherein the optical imaging device is capable of receiving an optical image of a population of cells of interest and converting said optical image into a digital representation and wherein
a) Said optical imaging device transmits said digital representation to said computer readable memory;
b) Said computer scans said digital representation at low-resolution for cell-like objects;
c) Said computer creates a listing, capable of being referenced by the computer at some later time, of each cell-like object in the digital representation thereby generating a list of salient, areas;
d) The computer chooses a salient area from the first element in said list of salient areas;
e) The computer retrieves the portion of said digital representation which contains at least said salient area for analysis and performs digital processing on said portion of the digital representation so as to identify, locate and store within said computer readable memory the location of at least one probe present in said portion of the digital representation;
f) The computer then chooses the next element in said list of salient areas, performing step e) above;
g) Step f) is repeated until a sufficient number of salient areas have been analysed, said sufficient number determined at the option of the computer system or by intervention of a human operator;
h) The system uses the location or at least one probe in each of the analyzed salient areas to determine the relationship of probes within each salient area; and
i) Said relationship of probes are used to generate a set of population statistics that may be used for analyzing the condition of said cells of interest.

The present invention also provides for a method of immobilizing cells in a microfluidic channel and preparing said cells for use in cellular analysis comprising
Taking a population of cells of interest, suspended in a fluid;
Filling a microfluidic channel with said population of cells of interest suspended in a fluid; and
Raising the temperature of said microfluidic channel to 55-95° C. for a period of time sufficient to allow immobilization of a portion of said population of cells of interest to said microfluidic channel;
Wherein time sufficient to allow immobilization of a portion of said population of cells of interest is determined by intervention of a human operator as the immobilization of a certain portion of cells of interest, either in terms of net number of cells immobilized, or alternatively as a percentage of total cells present in said population of cells of interest.

In one aspect, the cellular analysis is FISH.

In another aspect, the fluid is a buffer suitable for maintaining the size and shape of the individual cells making up the population of cells of interest. In a further aspect, the fluid is 1× Phosphate Buffered Saline (PBS).

In another aspect, the temperature is raised to 75-85° C. In a still further aspect, the temperature is raised to 75-85° C. for a period of 10 minutes.

The present invention provides for a method of increasing the portion of cells of interest immobilized within a microfluidic channel comprising
Having at least one region within said microfluidic channel with a course surface;
Taking a population of cells of interest, suspended in a fluid;
Filling a microfluidic channel with said population of cells of interest suspended in a fluid so as to allow a portion of said population of cells of interest to come into fluid contact with said course surface of said microfluidic channel;
Raising the temperature of said microfluidic channel to 55-95° C. for a period of time sufficient to allow immobilization of a portion of said population of cells of interest to said microfluidic channel;
Wherein time sufficient to allow immobilization of a portion of said population of cells of interest is determined by intervention of a human operator as the immobilization of a certain portion of cells of interest, either in terms of net number of cells immobilized, or alternatively as a percentage of total cells present in said population of cells of interest; and

The present invention provides for an apparatus for performing cellular analysis comprising
A first access port/well;
A second access port/well; and
At least one microfluidic channel;
Wherein said first access port/well is in fluid communication with said second access port/well by means of said at least one microfluidic channel;
And wherein said at least one microfluidic channel is of dimensions no greater than 110 μm×620 μm×100 mm.

In one aspect, the dimensions of the microfluidic channels are 55 μm×310 μm×50 mm.

In another aspect, the first access port/well and second access port/well each have a volume of 1.5 μL.

In another aspect, said microfluidic channels and said first and second access ports/wells are formed by etching a planar glass surface.

In another aspect, said microfluidic channels and said first and second access ports/wells are formed between the interface of a planar surface glass chip and a moulded, flexible plastic. In a further aspect, the flexible, moulded plastic is PDMS.

The accompanying description illustrates preferred embodiments of the present invention and serves to explain the principles of the present invention

### BRIEF DESCRIPTION OF THE FIGURES

FIG. 1 shows a representation of the insertion of FISH probes into DNA, from left to right: the complete probes and DNA strings, denatured genetic material, probe hybridization and a labelled chromosome.

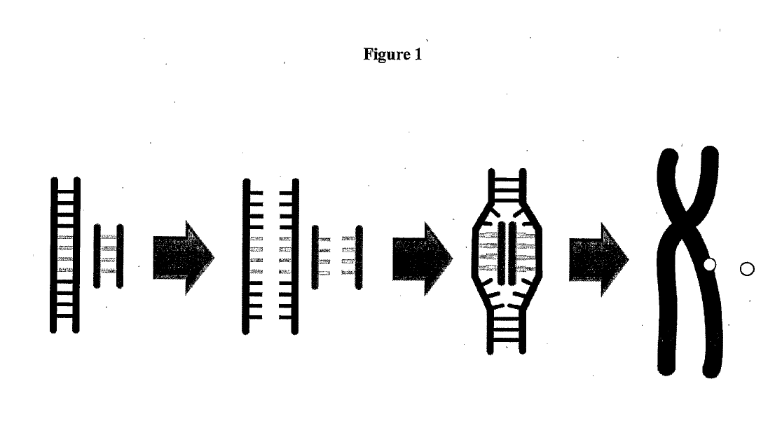

FIG. 2 shows the break-apart and dual-fusion probe based FISH analysis techniques for detecting chromosomal translocations.

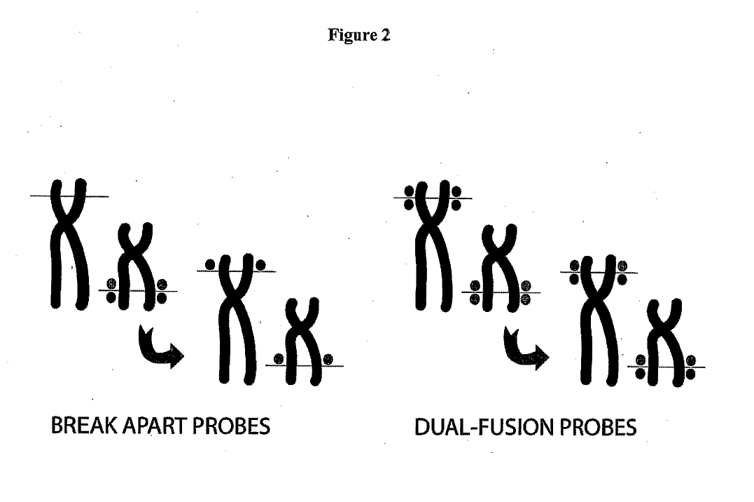

FIG. 3 shows the percentage of cells immobilized on the bottom surface of the microchannel at various temperatures;

FIG. 4 shows (a) a fluorescence image of microchannel after completing a FISH experiment on a microchip array with a hybridization time of fourteen hours, (b) an expanded image of cells from the channel that illustrate the ability of microchip-based FISH to distinguish translocated cells (KMS-12-BM) from cells having normal chromosome patterns, (c) a picture taken from conventional interphase FISH protocol completed on a patient sample with a microscope slide after fourteen hours of hybridization;

FIG. 5 shows the signal-to-noise ratio (hybridization efficiency) versus the hybridization time with a constant probe concentration; using the RAJI cell line and the break apart probe;

FIG. 6 shows the signal-to-noise ratio (hybridization efficiency) versus the hybridization time with a constant probe concentration; using the RAJI cell line and the break apart probe;

FIG. 7 shows two schematics for microfluidic chips used for interphase FISH analysis, (a) Microchip array used to perform the microchip-based FISH protocol, (b) Sample cross-section of a microchannel in the microchip array, (c) Combined mask layouts and dimensions of circulating microchip, (d) Cross section of a valve in closed position, (e) Sample cross section of a valve in open position;

FIG. 8 shows the conceptual system overview of the computational vision system of the present invention;

FIG. 9 shows a summary of the Type 1 center-surround ganglion filter response to differing input signals;

FIG. 10 shows an example of k-means performance on a broken pair as compared to that of brute-force clustering;

FIG. 11 shows sample CCI-01 analysis, a very sharp cell with two very close matched pairs and a natural background with system clustering decisions (top right), compared to the initial cell (top left) and the middle row of images indicating saliency processing output, while the bottom row of images indicates Cythe geometry extraction (vertical image columns indicating the respective color channel);

FIG. 12 shows sample AML-00 analysis, a cell with two matched pairs and the background has been cropped to black around the cell region with system clustering decisions (top right), compared to the initial cell (top left) and the middle row of images indicating saliency processing output, while the bottom row of images indicates Cythe geometry extraction (vertical image columns indicating the respective color channel);

FIG. 13 shows sample AML-02 analysis, a cell with two matched pairs and a partially cropped background (manual cropping with system clustering decisions (top right), compared to the initial cell (top left) and the middle row of images indicating saliency processing output, while the bottom row of images indicates Cythe geometry extraction (vertical image columns indicating the respective color channel);

FIG. 14 shows AML-01 analysis, a difficult cell with one broken and one matched pair, low contrast and with a natural background with system clustering decisions (top right), compared to the initial cell (top left) and the middle row of images indicating saliency processing output, while the bottom row of images indicates Cythe geometry extraction (vertical image columns indicating the respective color channel);

FIG. 15 shows sample CCI-02 analysis, a cell with an irregular shaped nucleus, one broken pair and one matched pair, very sporadic natural background with system clustering decisions (top right), compared to the initial cell (top left) and the middle row of images indicating saliency processing output, while the bottom row of images indicates Cythe geometry extraction (vertical image columns indicating the respective color channel);

FIG. 16 shows an example of the preferred embodiment of the computational vision system, wherein a low resolution scan and identification of the salient areas occurs along with identification of probe information.

FIG. 17 shows a sample vision system output and a comparison between the labelling of the system and the labelling of a human fish expert for a p53 deletion case and a IgH break apart translocation case.

### DETAILED DESCRIPTION OF THE PRESENT INVENTION

While particular embodiments of the present invention have been described in the foregoing, it is to be understood that other embodiments are within the scope of the invention and are intended to be included herein. It will be clear to any person skilled in the art that modification of and adjustment to this invention, not shown, are possible without departing from the spirit of the invention as demonstrated through the exemplary embodiments. The invention is therefore to be considered limited solely by the scope of the appended claims.

It should be noted that the computational procedures contemplated by the present invention are applicable to most forms of FISH or other types of cellular analysis, so long as the analysis results in a set of coded image channels. It is contemplated that the present invention applies to both metaphase and interphase FISH analysis, as well as spectral karyotyping and other methods involving labelling chromosomes with detectable probes, and methods involving “painting” parts of a cell with detectable probes to test for the presence of any of a wide variety of cellular markers, for example but not limited to, using tagged antibodies, chemicals that selectively localize in cells or ligands for particular receptors. As part of the present invention, the described methods and procedures are contemplated as having application in diagnostics or research, where a detectable probe is applied to a cell or tissue and wherein the absolute position, relative position, luminosity or other characteristic of the probe is relevant. Though the present invention utilizes nucleotide based-probes, the methods of the present invention are applicable to antibody based staining, receptor-ligand based staining or probing, or such other means for labelling and visualizing the presence of an atom, molecule or compound on a cell or tissue.

As well, it should be noted that the present invention is not contemplated as being limited to the chip design, manufacture or structures disclosed herein as non-limiting examples, except where are specifically noted. One skilled in the art would recognize that the formation of the microfluidic channels and ports/wells contemplated by the present invention can be undertaken using a number of materials, devices and procedures.

Recent research has shown that the detection of one particular mutation—the t(4;14)(p16;q32) translocation—has significant relevance to predicting patient survival rates and receptivity to common treatment methods (Gertz, M. A. et al., Blood, 106(8):2837-2840 (2005); Dewald, G. W. et al., Blood, 106(10):3553-3558 (2005); Jaksic, W. et al., J. Clin. One. 23(28):7069-7073 (2005)). Patients with the t(4;14) translocation have shorter survival times (18.8 months as opposed to 43.9 months), will show quicker relapse times, and receive minimal benefit from traditional stem cell transplantation and chemotherapy. As such, it is important to determine the receptivity of the patient to chemotherapy. AS well, transplantation is extremely hard on the patient and should be avoided if it can be shown to be ineffective for a given genetic type (Gertz, M. A. et al., Blood, 106(8):2837-2840 (2005)). Patients with t(4;14) can therefore be identified as an example of ideal candidates for novel emerging therapeutic procedures.

The present invention provides the first microfluidic platforms capable of performing rapid interphase FISH analysis. Peripheral blood mononuclear cells (PBMC) were used for the detection of chromosomal abnormalities in malignant cells from patients with MM. The design of the analysis system of the present invention has the approximate dimensions as the conventional microscope slide used in FISH, but is capable of performing analysis on a multiplicity of samples concurrently with reduced reagent usage per sample. IN a preferred embodiment, the analysis system of the present invention is capable of performing analysis on up to 5 samples concurrently, and in an even more preferred embodiment up to 10 samples concurrently. Additionally, in a preferred embodiment, the analysis system of the present invention is capable of performing analysis on a multiplicity of samples using ⅕^{th} the reagent usage per sample, and in an even more preferred embodiment 1/10^{th} the reagent usage per sample. A variety of microfluidic chip dimensions are contemplated as consistent with the present invention with variations in size shape and thickness. Various microchip implementations, as described herein, were capable of reliable immobilization of the target cells, enzymatic treatment of the target cells, controllable addition of DNA probes, and enhanced hybridization. This facilitated rapid FISH analysis. In the system of the present invention, the microchip-based FISH was capable of completion in hours as opposed to the days required by the conventional approach and was more cost effective in terms of reagent consumption and labor.

### Conventional Interphase FISH

In a standard interphase FISH analysis cells are immobilized for observation (Fluorescence In-Situ Hybridization: A Practical Approach, Beatty, B. et al., Oxford University Press (2002); Introduction to Fluorescence In Situ Hybridization: Principles and Clinical Applications, Andreeff, M., New York: Wiley-Liss (1999)). One way to immobilize the cells under investigation is by cytospinning them onto a glass microscope slide. Typically ˜30,000 cells are spun onto a microscope slide, of which ˜8000 cells remain adhered after a FISH experiment, roughly 20%. The slides are then left at room temperature for a few days to “age”, which results in better hybridization signals and stronger adhesion of cells (Fluorescence In-Situ Hybridization: A Practical Approach, Beatty, B. et al., Oxford University Press (2002)). Next, proteinase K digestion is performed to remove cytoplasmic and chromosomal proteins and RNA, improving accessibility to the chromosomal DNA. Following the digestion, the chromosomal DNA is dehydrated and fixed with a series of ethanol treatments that enhance the attachment of chromosomes and nuclei to the slides. The DNA probes are then added onto the slide and a coverslip is placed and sealed with rubber cement to prevent evaporation. Both the probe and chromosomal DNA are denatured (split into single stranded DNA) by heating the slide to a temperature of 75° C. for 5 minutes. The slide temperature is reduced to a temperature of 37° C. and after time (typically overnight), hybridization of probe DNA to the chromosomal DNA will be evident. To reduce any cross-hybridization (non-specific binding), the slides are rinsed with a post-hybridization solution. The cells are then analyzed and classified (discussed below) by fluorescence imaging to yield a diagnosis (Netten, H. et al., Cytometry, 28:1-10 (1997)). Depending on where the probe hybridizes along the chromosomal DNA, detection of various chromosomal abnormalities including amplifications, deletions, insertions, and translocations is possible, giving interphase FISH techniques a broad range of capabilities for diagnostic testing.

One type of probe commonly used to detect translocations associated with MM is the “Break Apart” probe. Conceptually, the chromosomal locus of interest is labelled with two different fluorophores flanking the spot where the break point is located. When a translocation occurs, one of the colors is left on the original chromosome, while the translocated portion with the other color is found on another chromosome. Thus when imaging a cell, if the two colored dots are close, there is no translocation, but when the colored dots are far apart (greater than two signal diameters), a translocation exists.

Current clinical FISH visualization systems are large and expensive (in the neighbourhood of $200,000 including detection software). While the optical systems are accurate and produce excellent high-resolution images, a human expert is still needed to visually identify the presence of translocations in a sample cell population. At least two-hundred cells must be analyzed to detect the absence or presence of a particular translocation. Given the presence of difficult or damaged cells, or a low frequency of malignant cells in a population, sometimes more than five-hundred cells must be analyzed per sample. The larger the sample population, the greater the statistical relevance of the results. The results are categorized by the ratio of abnormal probes configurations (broken pairs, for instance) present over the ratio of abnormal probe signals detected in a healthy baseline patient profile. Through rapid automation, these large samples are scanned without human intervention, freeing up the expert's time and facilitating the move to miniaturized diagnostic technologies. The present invention provides an automated system capable of detecting both break-apart and dual fusion probe approaches, as well as any other pattern that identifies numerical or structural abnormalities from a FISH-labelled cellular image.

### Immobilization of Cells

One of the most common methods of immobilizing cells for FISH is by cytospinning them onto a microscope slide surface; however, this approach is not feasible in sealed microchannels. Other techniques have been researched for cell immobilization including physical absorption, covalent binding, ionic binding, physical entrapment, dielectrophoresis, and entrapment in gels (Maruyama, H. et al., Analyst, 130:304-310 (2005)). Of these methods, physical absorption is the simplest technique to implement on-chip, but the immobilization strength is comparatively weak. Initially, physical absorption was attempted, by loading the channels with cells and allowing them to settle and immobilize. With a channel height of 20 μm, the cells clustered together and momentarily clogged the chip, preventing further reagent flow. However, they did not adhere and when vacuum was applied they were completely removed. Likewise, Gaver et al. (Gaver, D. P. et al., Biophys. J., 75:721-733 (1998)) performed a theoretical study on cell adhesion in a microchannel by varying cell size, channel height and flow rate. As the cell size became comparable to the channel height, adhesion rates dropped by a significant amount (Gaver, D. P. et al., Biophys. J., 75:721-733 (1998)).

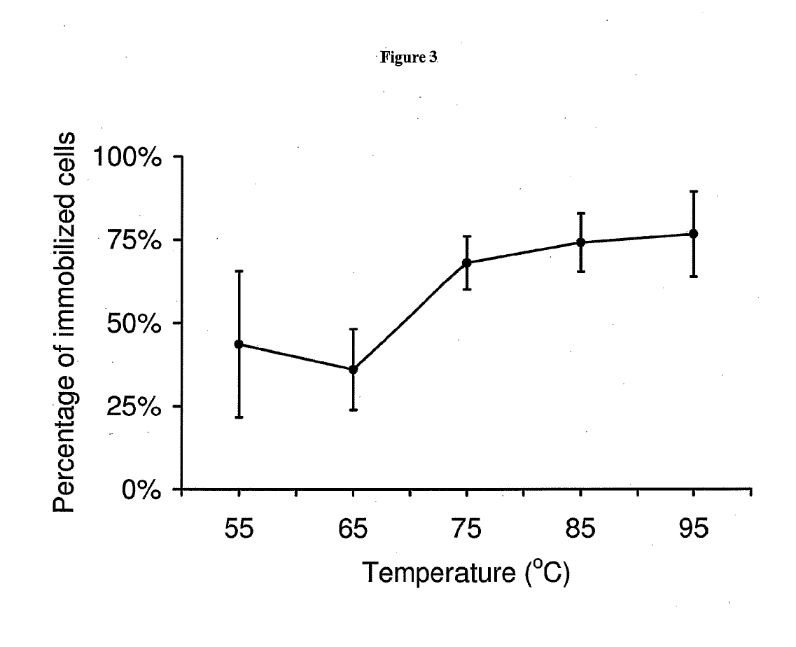

The minimum channel height for adequate cell immobilization while permitting reagent flow was in the range of 40-55 μm to implement physical absorption, but very few cells remained when the fluid phase was removed with vacuum. However, it was discovered that heating of the microchip resulted in an increased number of strongly adhered cells. To investigate this effect further, the chip was heated to various temperatures and three cell lines and cells from three ex-vivo patient samples were tested at each temperature. The temperatures ranged from 55° C. to 95° C., as very little adhesion occurred below 55° C. and any temperature above 95° C. was incompatible with later steps in the FISH protocol. Adhesion was assessed by adding the tested cells, suspended in 1×PBS, to the channel by capillary force and counting the initial concentration. The temperature treatment was applied for 10 minutes and the chip was returned to room temperature at which point the remaining solution was removed by vacuum. The channels were then imaged to count the remaining cells. FIG. 3 shows the percentage of cells (from the initial concentration in the channel) that adhered to the channel walls at various temperatures presented as the average of three cell lines and three patient samples (ex-vivo MM PBMC) with standard deviations (n=18). Cells were added to the channel and the initial number was counted.

Following the heat treatments, vacuum was applied and the remaining adhered cells were counted. Adhesion increases with temperature and the standard deviations decrease, allowing a more repeatable percentage of cells immobilized. At 95° C. the cells begin to burst, imposing an upper limit. As the temperature increased, the average degree of immobilization increased, and the standard deviation decreased. The standard deviations were calculated from three images of each channel over six channels (three cell lines and three patients) or n=18; therefore, the decrease with temperature signifies less variability in the percentage of immobilization. The lower temperatures (55-65° C.) appeared to immobilize adequately, but when flows were introduced many of the cells were dislodged, indicating only a slight improvement in adhesion. However, at 75-85° C., the cells were strongly adhered while maintaining the cell morphology. Above 95° C., severe damage to the cell morphology was noted, to the extent that FISH analysis was not feasible. Therefore in the preferred embodiment of the present invention immobilization of cells in a microfluidic channel for use in FISH and other applications is effected through raising the temperature of the channel to 55-95° C., more preferably 75-85° C. for a period of time necessary to result in the immobilization of at least one cell in the microfluidic channel. In the preferred, but non-limiting, embodiment this time is 10 minutes. The methods to raise the temperature of the microchannel are known to those skilled in the art and include, but are not limited to, raising the temperature of the entire microfluidic chip, or a localized area, through radiative heating or through conductive heating using a resistive element.

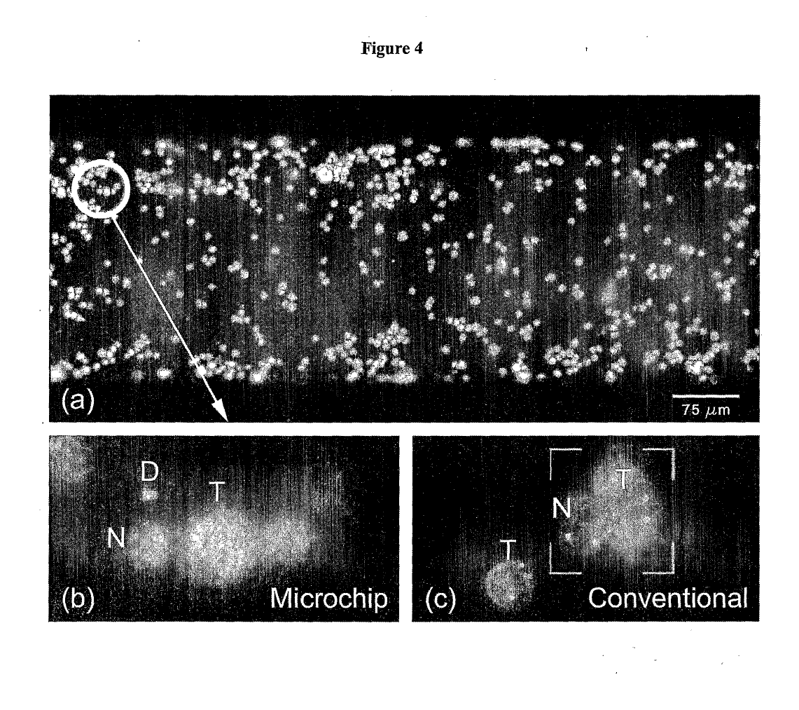

The fluorescence image in FIG. 4 illustrates a typical pattern of immobilization. The immobilization technique of the present invention, regardless of the sample, was able to reliably immobilize cells to cover at least 10% of the bottom surface area in a microchannel. Further, it was observed that almost 90% of the adhered cells were preferentially immobilized on the bottom surface of the microchannel (etched surface). The few cells that immobilized on the top surface or the side surfaces are evidenced in the fluorescence image (FIG. 4) by slight blurring. Cells immobilized on the side surfaces of the channels (noted by the apparent clustering) cannot be adequately assessed, and are consequently excluded from analysis in the preferred embodiment. It was also noted that cells adhered more densely to regions where the glass etch was rougher (coarse surface), such as the curved part of the microchannel (side surfaces). This phenomenon can be seen from the higher frequency of cells along each side of the channel in FIG. 4. Though not necessary to practise the present invention, it is hypothesized that this behaviour contributes to surface energy minimization along the curved portion of the channel. The adhesion to coarser areas was similarly observed when a section of the channel had a pattern of roughly etched glass, resulting in cells adhered more strongly and recreated the underlying pattern. It is therefore contemplated by the present invention to surface tailor microfluidic channels for site-specific immobilization.

The temperature immobilization of cells eliminated the use of specialized equipment, namely the cassette cytospin centrifuge. It was also observed that the temperature “aged” the cells, resulting in increased hybridization and signals without further treatment, such as the multiple days aging normally associated with the conventional method. The temperature immobilization also preserves more of the three dimensional cell structure, as discussed below.

### Direct FISH Implementation

Using a mixture of two cell types (normal and translocated), it was verified that the temperature-induced immobilization did not significantly affect the FISH protocol. After hybridizing the probe overnight, the cells were imaged within the microchannel on the fluorescence microscope and comparable performance to the traditional method was obtained. As shown in FIG. 4(a), a mixture of KMS-12-BM (bigger cells that harbour a 14q32 translocation) and ex-vivo cells from a patient (smaller cells) with a normal chromosomal complement were imaged. As shown in FIG. 4(b), debris, marked D, was more commonly observed in microchip-based FISH. As shown in FIGS. 4(b) and 4(c), the “normal” cells, marked N, have red and green probes close together, as discernible by paired red and green dots, signifying the absence of a translocation. The malignant cells, marked T, have at least one probe clearly broken away from the counterpart color, indicating a translocation. Both FIG. 4(b) and FIG. 4(c) were representative images of their respective methods and either image was readily interpretable. As shown in FIG. 4, normal cells were clearly distinguishable from malignant cells. Furthermore, the microchip-based FISH used 1/10th the probe, thus reducing the cost substantially as the probes are relatively expensive (for example, $90/test reduced to $9/test). It will be clear to one skilled in the art that further cost reductions are possible. Subsequently, tests on a variety of cell lines and patient cells in combination with various probes to assess performance and ensure robustness of the protocol were performed. Table 1 summarizes the combinations of cells and probes performed with microchip-based FISH, confirming the stability of the developed protocol with a variety of samples.

TABLE 1
Microchip-based interphase FISH with multiple
cell and examples of probe combinations.

| Cell | Probe* | Detected** |
|---|---|---|
| RAJI line | LSI IGH dual color, break | 14q32 |
| | apart | translocations |
| KMS12-BM line | LSI p53 and CEP17 D17Z1 | 17p13.1 deletion |
| KMS18 line | LSI D13S319 | 13q14.3 deletion |
| Patient 1 | LSI IGH/FGFR3 dual color, | t(4; 14)(p16.3; q32) |
| | dual fusion | |
| Patient 2 | LSI IGH dual color, break | 14q32 |
| | apart | translocations |
| Patient 3 | LSI D13S319 | 13q14.3 deletion |
| 80% KMS12-BM + | LSI p53 and CEP17 D17Z1 | 17p13.1 deletion |
| 20% RAJI | | |
| 80% Patient 1 + | LSI IGH/FGFR3 dual color, | t(4; 14)(p16.3; q32) |
| 20% KMS18 | dual fusion | |

After verifying the robustness of the microfluidic chip protocol, samples were tested from multiple myeloma patients for some of the most common chromosomal abnormalities present in this disease. Bone marrow mononuclear cells were tested from eleven patients and PBMC in one patient diagnosed with plasma cell leukemia. Commercial probes were used along with locally prepared probes and tested each patient for 9 different chromosomal abnormalities: deletion of 13q14.3, any translocation in IgH locus (14q32), translocation t(4;14)(p16;q32), translocation t(11;14)(q13;q32), translocation t(14;16)(q32;q23), amplification of 1q21, deletion of p53, amplification or deletion of 19q13.4 and amplification or deletion of 5q33.2-qter. The results obtained using on chip FISH for each patient matched exactly the results obtained with the conventional FISH method on microscope slides.

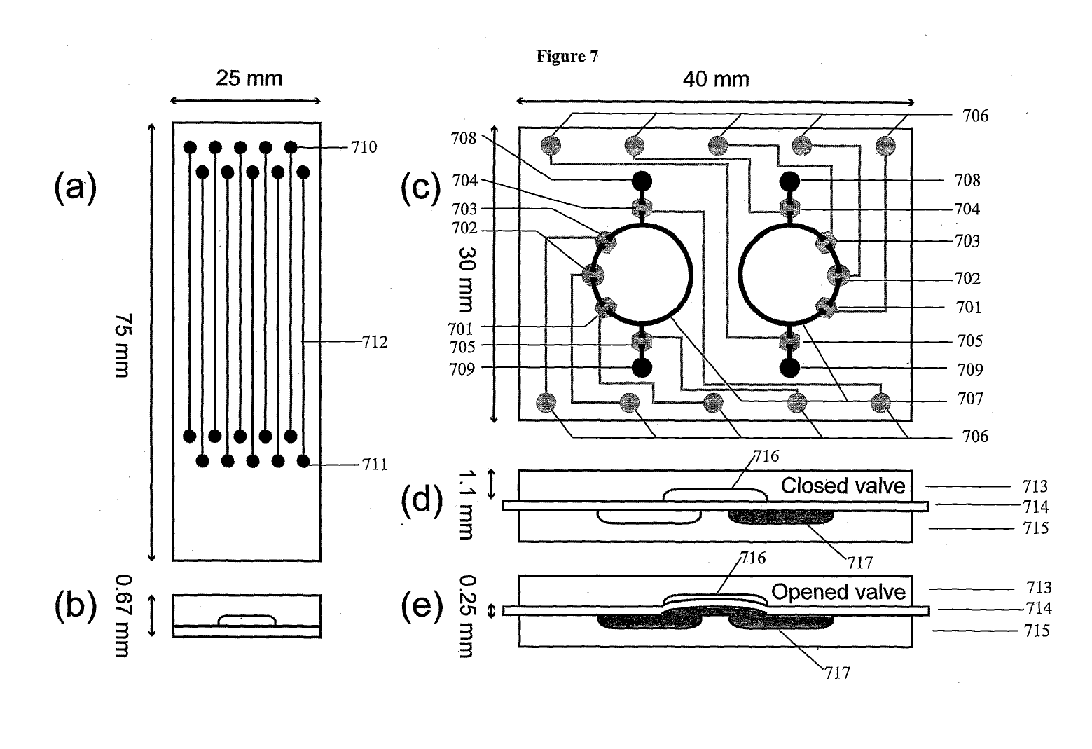

FISH was also performed with a polydimethylsiloxane (PDMS) version of the straight channel chip shown in FIG. 7. These microchips were created using the soft-lithography approach (Ng, J. M. K. et al., Electrophoresis, 23(20): 3461-3473 (2002)). The PDMS portion was bonded the to a glass substrate; PDMS top with channels bonded to a thin glass coverslip. The FISH protocol was identical for the PDMS chip and glass straight channel chip. Practicing the invention on the PDMS chip provides for a disposable method wherein each sample is tested on a different chip that is never reused.

### Diffusion Studies

Picks relation for diffusion provides us with the following equation describing how far a molecule will diffuse with a given diffusion coefficient (D), dimension (n=˜1D) and time (t).

r=\sqrt{2nDt}

Using this diffusion analysis with values available in the literature, one can calculate the time required for probes to travel to the vicinity of the cell and hopefully enter and hybridize. The following table summaries key distances in a FISH experiment. It becomes clear that probes not directly over cells are not utilized in a typical FISH experiment (hybridization time of 16 hours). For this reason, the present invention provides for a microfluidic channel which confines probe directly over the immobilized cell region, yielding the optimal utilization of expensive probe.

TABLE
Diffusion times

| | Distance | | | | |
|---|---|---|---|---|---|
| Parameter | (1D) | | Frag. Size | Time | |
| Cell-to-cell space | 40 | um | 100-400 bp | 20-68 | seconds |
| Over-night hybridization | 1 | mm | 100-400 bp | 3.5-11.8 | hours |
| Shandon radius (cell | 3 | mm | 100-400 bp | 31-105 | hours |
| region) | | | | | |
| Coverslip (total probe) | 6 | mm | 100-400 bp | 5-17 | days |

### Hybridization Studies

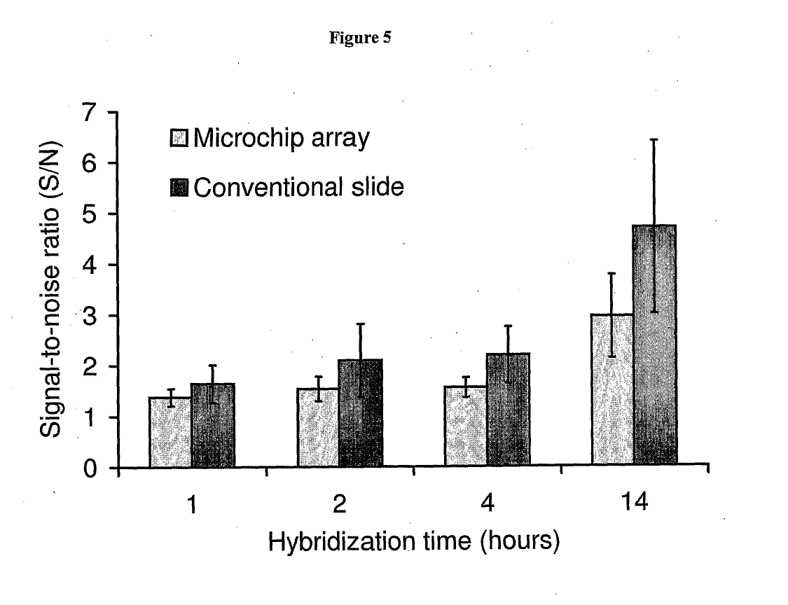

Although the probes hybridized adequately, it was noted that the microchip implementation had a slightly lower signal-to-noise ratio than the conventional microscope slide-based FISH. To study this difference further, a series of time-based diffusion hybridization experiments were performed for one, two, four, and fourteen hours, as shown in FIG. 5. The one and two hour hybridizations for both microchip and slide methods yielded many cells that were hybridized; however, the degree of hybridization was heterogeneous, with many cells having little signal. Although the one and two hour experiments provided detectable chromosomal patterns for those cells having adequate signal, many cells did not have adequate signal. However after four hours, cells had a more homogeneous degree of hybridization in which most cells had adequate signal. One trend seen throughout each hybridization experiment was that the microchips had higher levels of background noise than the slides. After imaging the microchip arrays and microscope slides on a confocal microscope, it was determined that the cells advantageously retained more of their 3-D structure on the microchips, being less “flattened” with the temperature immobilization than with the cytospinning protocol used conventionally. Attachment of cells by cytospin centrifugation is known to cause substantial flattening of the nuclei (Fluorescence In-Situ Hybridization: A Practical Approach, Beatty, B. et al., Oxford University Press (2002)). The slight signal-to-noise variation shown in FIG. 5 is attributed to the methods of immobilization employed. The temperature-induced procedure preserved more of the cell structure, thereby creating higher levels of background. Nevertheless, the images were readily analyzed by a trained technician experienced in FISH or by computer vision system of the present invention. Whether by trained technician or the tolerance-based computer method, the images were comparable in quality to images from conventional FISH. With the microchip-based FISH protocol established, electrokinetic and mechanical pumping of the probes to increase the hybridization speed was investigated. Thus a further aspect of the present invention is a method to immobilize cells on a reusable or disposable microfluidic chip while maintaining 3-D structure for analyses that require assessment of physical localization within a cell or to determine on chip the physical relationship between the molecules and/or structures detected by two or more colored tags.

### Hybridization Enhancements

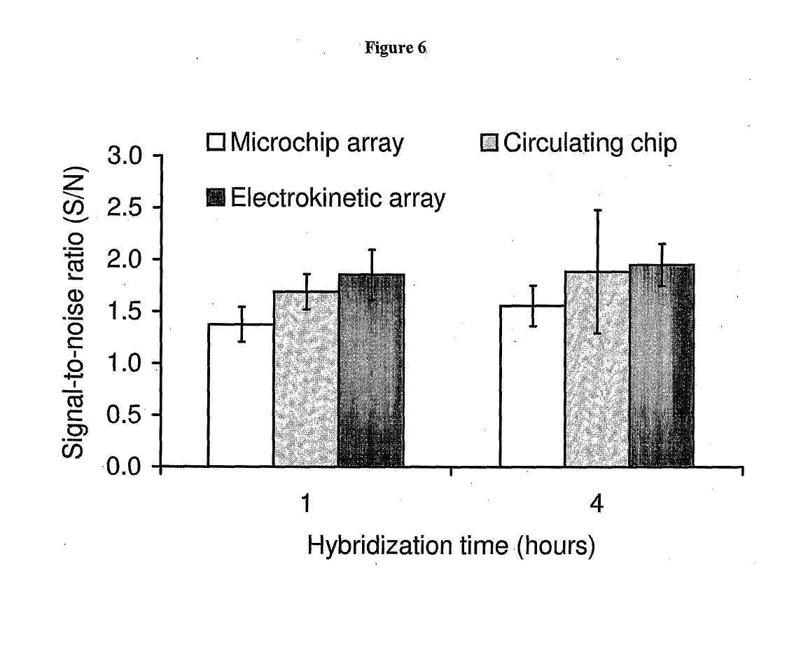

Typically, DNA microarray technologies attain order of magnitude decreases in the hybridization time when the solution is agitated (Bynum, M. A. et al., Anal. Chem., 76:7039-7044 (2004)); however, other barriers to hybridization within a largely intact cell present a substantially more complicated situation (Fluorescence In-Situ Hybridization: A Practical Approach, Beatty, B. et al., Oxford University Press (2002); Introduction to Fluorescence In Situ Hybridization: Principles and Clinical Applications, Andreeff, M., New York: Wiley-Liss (1999)). Although interactions with intracellular components slow hybridization inside the cell, microchannels minimize the diffusion distance for probes outside the cell. The diffusion distance is minimized by confining the width and height of the microchannel, which are tunable to accommodate the procedures being performed, while cycling the probe solution along the channel. Pneumatic or electrokinetic pumping provides a good strategy for accelerating hybridization, with a pumping cycle to ensure a significant concentration of probes local to the cells, followed by a one minute pause allowing enough time for probes to diffuse into the cells and hybridize. To estimate the “pause” time, the time required for a probe to diffuse from the top of the channel to the bottom was calculated using Einstein's diffusion equation (Vanderhoeven, J., et al., Electrophoresis, 26:3773-3779 (2005)). The DNA diffusion coefficient used, 1.0×10⁻⁷ cm²/s, was consistent with commonly used values in typical DNA microarray analysis (Smith, D. E. et al., Macromolecules, 29:1372-1373 (1996); Sorlie, S. S., Macromolecules, 23:487-497 (1990); Nkodo, A. E. et al., Electrophoresis, 22:2424-2432 (2001); Stellwagen, E. et al., Electrophoresis, 23:2794-2803 (2002)) for similar strand lengths. As illustrated in FIG. 6, these agitation methods gave ˜30% improvements in the signal-to-noise ratio. This corresponds to a four-fold decrease in hybridization time, as the signal-to-noise ratios at one hour with pumping are equivalent to or better than the diffusion-based hybridization at four hours. The present invention contemplates other circulation methods as giving rise to the increased benefits observed with electrokinetic and pneumatic pumping.

It should be noted that the homogeneity of probe hybridization when employing mechanical pumping or electrokinetic transport was similar to diffusion-based experiments in that the one hour hybridizations were heterogeneous and the four hour hybridizations were more homogeneous. In the electrokinetic experiments, the channel walls near the electrodes maintained a high level of background fluorescence and cells closer to the electrodes appeared to have localized areas of non-specifically bound probes. Although the probe is not fully utilized in these regions, the chip and instrumentation will allow considerable flexibility in developing improved methods to optimize probe usage. Similar results are achievable with probe dilutions of as little as 1/60, albeit with lower hybridization speeds.

### Computational Vision System for FISH Applications

The present invention provides a computational vision system to effectively extract relevant, information from fluorescent in situ hybridization images. Though contemplated to form part of the method, system and apparatus of the present invention; it is also capable of use with cellular analysis systems, or in substitution of an expert human observer in cellular analysis systems. To be effective as an analysis pipeline, the system must be able to:
Detect the location and size of whole cells
Decide if a cell is worth evaluating
Detect the location of FISH probes within a cell
Group the probes into accurate clusters
Calculate the distance between the probes of each cluster
Apply diagnostic rules to the probe clusters
Return a comprehensive list of cell and probe statistics

The computational vision system of the present invention is able to take a multi-channel image and extract the features of interest in the form of geometric feature clusters. This is a problem of saliency—generating a system to determine the features of interest in a scene and directing system attention to these features. The prior art describes saliency processing; computational systems that are able to receive an input image, expand the input image into a series of color and feature selective maps, recombine the maps into a general saliency map, and then direct attention to the salient features of the input image (Walther, D. et al., Biologically Motivated Computer Vision, Second International Workshop (BMCV2002), 472-479 (2002); Itti, L., et al., IEEE Trans. on Pattern Analysis and Machine Intelligence, 20(11):1254-1259 (1998); Sha'ashua, A. et al., IEEE Conf., (1988)). The prior art has disclosed ways to increase the system's ability to boost recognition ability using color-difference saliency information (Van de Weijer et al., IEEE Trans. on Pattern Analysis and Machine Intelligence, 28(1):150-156 (2006)). While the prior art has shown it is possible to use these methods in a FISH environment, there have been no specific demonstrations of vision-system effectiveness for FISH methods in specific, and cellular analysis in general, is applicable to multiple myeloma or other cancer related translocations and integration with microfluidic devices has not been demonstrated (Theodosiou, Z. et al., Cytometry A, 71A:439-450 (2007)).

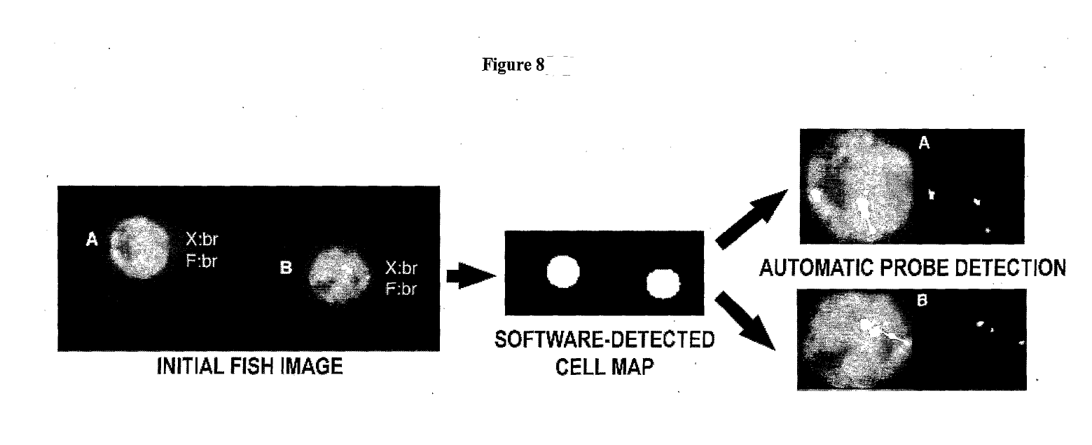

The computational vision system of the present invention is comprised of three stages: a cell-scale optical processing stage, a probe-scale optical processing stage, and an information extraction stage. These stages are connected by a computational framework that passes processing information between stages and maintains localization relationships between scaled image copies. The system takes as input a multi-channel FISH image containing at least one, and in a preferred embodiment at least 500, and in an even more preferred embodiment at least 1000, FISH labelled cell(s), the expected radius of the probe fluorescence, the expected radius of the target cells, and the level of background inhibition desired for each of the channels. It returns a comprehensive list of cell and probe locations, the relationships and distances between probes for each cell, and a diagnostic/statistical summary of the entire analyzed cell population. This system-level behaviour is summarized in FIG. 8, shown using real processing information from a preferred embodiment of the system.

The computational vision system of the present invention first decomposes the input image into a set of color paths. Because of the nature of the input data (as one probe type is encoded on one and only one image color channel) the computational vision system of the present invention is able to employ a much smaller architecture than the saliency systems used by full computer vision applications. As such, the present embodiment of the invention uses only the red, green, and blue color channels to determine region saliency; however, additional channels can be included or substituted. The present invention can also function when given only red and green color channels—a third channel can be created by adding both the red and green channels to form a single grey-scale image. The two probe types are detected individually from the red and green color channels, while the blue (e.g. DAPI nucleus staining) and/or the additive grey-scale channel facilitate the identification of the cell nucleus bounding area. Each channel is used to detect only a specific set of target objects. The prior art uses the modulation of object detection based on saliency (Navalpakkam, V. et al., Vision Research, 45:205-231 (2004)). The computational vision system of the present invention applies the opposite process, modulating saliency by object profile.

An image of the cell(s) of interest is received from an optical imager, of which any types are known in the art, and converted into a digital representation by means, such as, but not limited to, a CCD camera in digital communication with at least a computer readable memory and a digital system capable of converting the optical information received by the optical imager into a digital representation. Many such devices are known in the art. Once input images are split into color channels, the computational vision system of the present inventions applies an optical processing routine to each channel (described below). The channels are then normalized to standard grey-scale values (0-255) and their histogram is cropped (“inhibited”) to remove low-level background noise. The resulting filtered channel intensity maps represent the saliency information for the channels. These saliency images are then passed to a geometry extraction routine that decomposes each image into a hierarchy of geometric shapes. The extracted geometry is then parsed to create a list of labelled points. These points (representing cell and probe location, shape, size, and other geometric information) are clustered using a brute-force clustering algorithm. The computational vision system of the present invention then returns an array of cell locations, clustered probe locations and the distance measure between the probes of each probe pair/distance between pairs. As cell nucleus boundary information is detected during processing, the point list can optionally be cropped to include only probes found within detected cell nuclear boundaries.

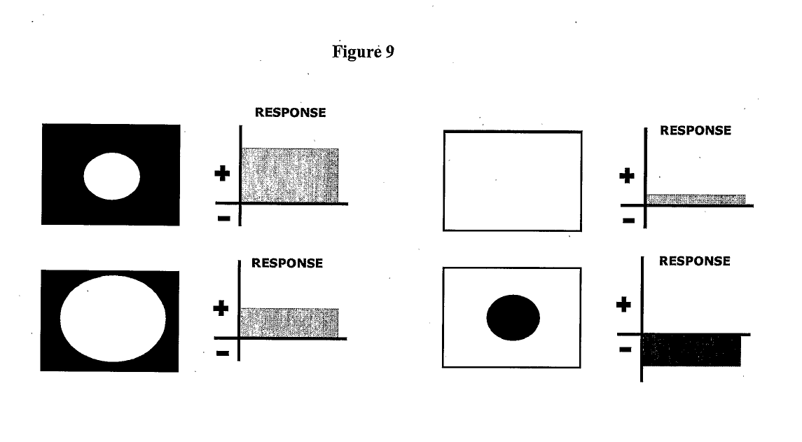

To effect the optical channel processing mentioned above (used to detect both cells and probes), the computational vision system of the present invention uses a regional processing method on each channel that is modelled after the low level processing of retinal ganglion cells in the biological eye—it applies a center-surround cell activation model to each pixel of the input region (Theodosiou, Z. et al., Cytometry A, 71A:439-450 (2007); Meister, M. et al., Neuron, 22:435-450 (1999)). The system uses two ganglion models: a Type 1 center-surround model that shows higher activation for light circles on a dark background, and a Type 2 center-surround model that responds best to a dark point on a light background. These cells are formulated using the Difference of Gaussians mathematical filter model (Computer Vision, Shapiro, L. G., & Stockman, G. C., Prentice Hall (2001); Meister, M. et al., Neuron, 22:435-450 (1999)). The input response of the Type 1 model is summarized in FIG. 9. AS the input shape begins to approximate the filter model shape, excitation increases. For all other shapes excitation decreases.

The input parameter for the two center-surround filter models (Type 1 and 2) is a receptive field radius that controls the size of the region used to determine their excitation level. The Type 1 model effectively enhances the contrast of the image and shows high excitation on circular intensity regions that loosely fit a Gaussian profile (such as a healthy cell nucleus or a probe). Type 2 instances smooth out image features if applied with high receptive field sizes, successfully homogenizing large feature regions; this is useful in determining cell boundaries without a labelled DAPI blue channel in the input image—a homogenized red and green channel can be used in place of a labelled DAPI channel. As such, the Difference of Gaussians (DOG) algorithm used in the present invention is described as follows.

The model for the retinal ganglion receptive field was based on a Difference of Gaussians model (Meister, M. et al., Neuron, 22:435-450 (1999)). The activation A(x,y,i,j) at each point in a given filter's receptive field is derived from the combination of two Gaussian plots centered on the middle of a receptive field of radius R. The pair (x,y) represents to center of the receptive field in the coordinates of the image space I, while the pair (i,j) indicates the relative location inside the receptive field (based on the field origin (x,y)). The intensity L(i,j) represents the input pixel amplitude to the Gaussian pair.

The distance D from the center point (x,y) to the sample point is calculated radially as:

D=\sqrt{(i−x)²+(j−y)²}

Thus the activation for each point is shown as:

$$A ( x , y , i , j ) = L ( i , j ) * ( {k}_{c} * {e}^{- \frac{{D}^{2}}{2 * {r}_{c}^{2}}} + {k}_{s} * {e}^{- \frac{{D}^{2}}{2 * {r}_{s}^{2}}} )$$

The activation A(x,y,i,j) for the filter G at (x,y) is then tabulated as follows to determine the final filter output level:

$${G}_{( x , y )} = {\sum }_{\vert ( i , j ) \vert < R} A ( x , y , i , j )$$

For Type 1 fillers the following constants were identified experimentally to enforce a large distinction between the different activation states of the population. k_{c} is the Center Gaussian Amplitude, r_{c} is the Center Gaussian Radius, kₛ is the Surround Gaussian Amplitude, and rₛ is the Surround Gaussian Radius.
k_{c}=1.0
r_{c}=−0.2
kₛ=R/3
rₛ=R*2

Similarly, for Type 2 filters the following constants were identified:
k_{c}=−1.0
r_{c}=1.0
kₛ=R/2
rₛ=R*6

It is important to note that the Type 1 and Type 2 Gaussian parameters (deviation and amplitude) used in the present invention differ from the standard values in the prior art.

One important feature of undamaged cell and probe fluorescent signals is that they typically resemble bright circular points on a darker background region. This is important for probe and cell detection as it is improbable for noise artefacts to match the signal profile of a true cell or probe. This makes the Type 1 DOG filter a perfect match for the probe model. As a result, the Type 1 processing of the present invention effectively enhances primarily probe and cell objects without giving significant excitation to usual background features, intensity ridges such as the cell boundary, and natural image noise. The receptive field of the Type 1 filter is tailored to the observed or estimated (either manually or automatically) size of the cell and probe fluorescence in a given sample population of images—the radius of the filter should be slightly larger than the actual intensity radius of the probe(s), so that the probe(s) fit into the excitation region of the center surround cell. This is a case of model matching, and result in a saliency channel containing accentuated probe regions and inhibited image noise.

The normalized output of the Type 1 filter is then inhibited by a baseline value that may be determined individually for each probe color type to remove the remaining noise and re-normalized to the full intensity range. This process is designed to remove all low-level background information and ensure that the saliency information on all channels is represented in equivalent intensity units (in this case the standard 8-bit grey-scale range of 0 to 255, though other intensity descriptors—such as unit-scale floating point numbers—may be used).

An additional modification was needed to insure accurate image processing by the invention. It was found that image boundary effects lead to the detection of erroneous salient feature along the image boundary; the transition from image background to the space outside the image (typically represented as either undefined values or solid black) which produces a noticeable filter output. For this reason, an image pixel boundary approximation was used to extrapolate the image information outside the image boundary. If a pixel referenced by the DOG algorithm was past the boundary of the initial image (e.g. non-existent), the boundary pixel value of the image was used in place of a referenced pixel's null value. In the case of a single axis of the pixel exceeding a boundary value, the intensity value of the corresponding (projected) point at the boundary was used. In the case that both axes were out of bounds, the intensity value of the nearest image corner was used. This ensures that erroneous edges are not detected near the image boundaries, and prevents artefact generation if the image is cropped to black.

Once the saliency maps have been computed, normalized, and stored in a computer readable memory as images, they are converted to a hierarchal structure of geometric objects using Cythe, a parameter-based geometric extraction system (Pilarski, P. M., and Backhouse, C. J., Optics Express, 14:12720-12743 (2006)). Cythe segments the image, detects contiguous regions using a distributed communication scheme, and then models each contiguous region as a geometric shape with a computed width, height, pixel mass, and centroid.

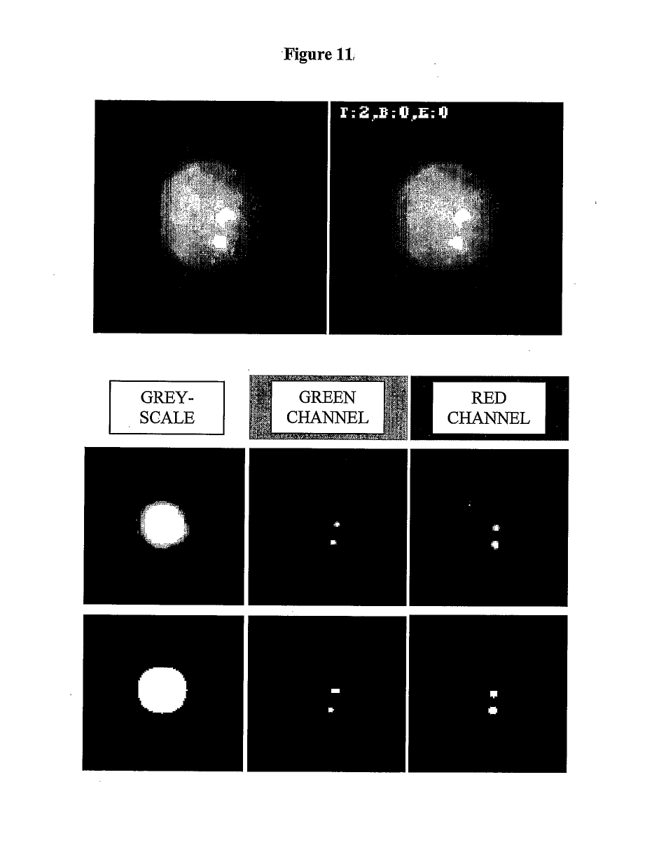

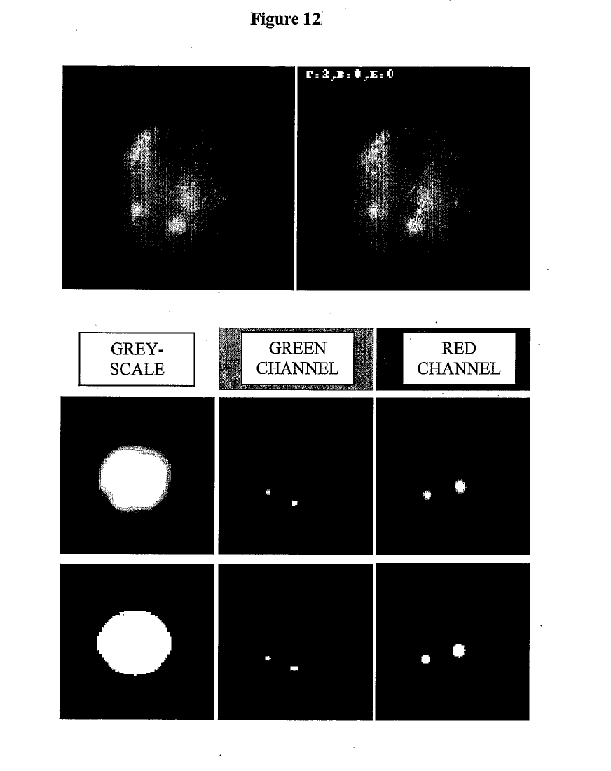

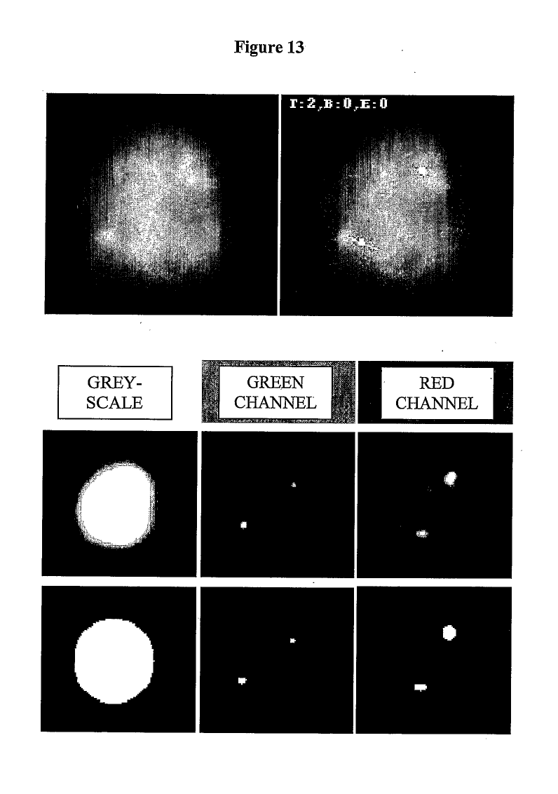

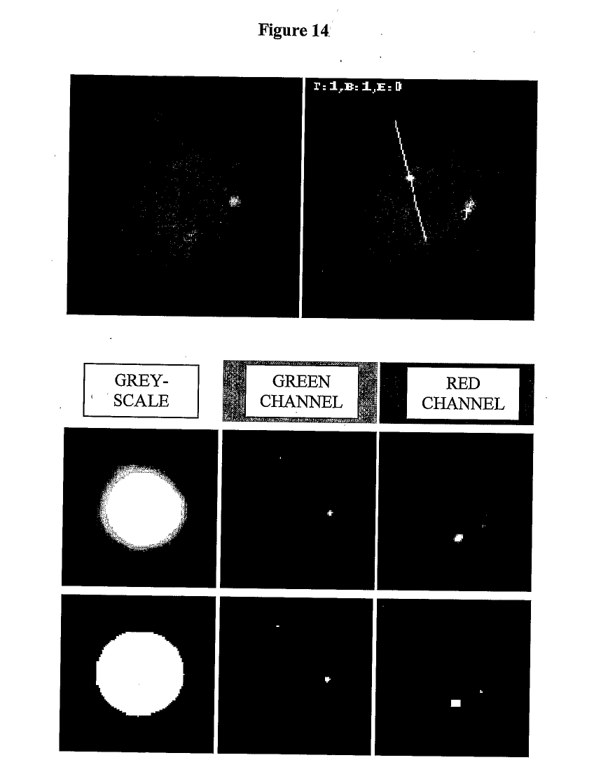

Initial attempts were made to sum the saliency information through operations in image space, but it was found that the probe clustering and analysis was much more effective in geometric space. As such, each detected saliency region (probe or cell) is stored in a hierarchal geometry framework containing the image, the labels for each region, and all identified image objects. In this case, the Cythe algorithm returns a geometric model for each of the salient points in each image channel. This information is made available to the computer vision system via a returned array of geometry objects. Cythe also performs additional low level noise removal and small group removal as part of its threshold segmentation and grouping procedures. The result of the Cythe geometry extraction on a set of saliency channels from the computational vision system of the present invention can be seen in the bottom row of FIGS. 11-15.

In the last stage of the computational vision system of the present invention, the system examines each detected cell region. Within each cell object boundary it clusters the detected member probe objects into pairs. The resulting cell and pair statistics are then evaluated for clinical merit.

First, the probe geometry output from the Cythe process is labelled according to channel type. This results in a list of probe points labelled with the color of the detected probe or probes. Optionally, this list can be cropped using the geometric cell model derived using the blue or greyscale saliency channel. If this option is employed, all probe points outside detected cell regions are ignored in the pair computation, as for the t(4;14) or del(p53) case, real chromosome pairs are typically not located outside the nucleus of a normal undamaged cell. Alternatively, a small buffer zone outside each detected cell boundary may be specified to include additional probes in pair computation.

The probe point list is then passed to a clustering algorithm. In this version of the system a brute-force pairing algorithm is used to group probes into pairs with minimal separation distance between member probes. Domain knowledge is used to limit the clustering problem to the formation of exact pairs and single outliers, with clustering preference given to the distance between probes. The nearest probes are clustered first, then the remaining probes in order of pair separation. In the event of a single remainder probe, it is put into its own cluster and labelled accordingly.

The brute-force clustering method calculates the edge lengths between all salient points in the geometric image space (in this example, salient points are stored as a list of labelled point objects, P). It then assigns point pairings based on the shortest detected distances. The two closest points are grouped into a pair, and removed from the point list. This process repeats until the point list P is completely empty. In the event that a single point is left in the point list, it is removed and added to its own group. Once all points have been clustered the algorithm computes the centroid for each pair and the average distance to the two member points. This information is used in the clinical classification of each pair (e.g. as either a joined or a broken probe set). The algorithm terminates with the return of a complete pair list of all probes in the observed field (cell). This is repeated for every detected cell object. As each cell under analysis should not have an unmanageable number of probes this algorithm is computationally tractable for FISH applications and gives highly accurate clustering pairs.

To deal with a wide range of analysis situations, the clustering system of the present invention may be used to only cluster points of opposite color (e.g. red-green pairings) or allowed to cluster any nearby points of any color label (e.g. red-red, green-green, red-green, red-blue, etc.) depending on the specific target application. In addition, for different cell analysis cases, threshold values may be set to allow small nearby probes of the same color to be treated as a single probe (e.g. to deal with the case of “split chromatides”). As the probe list is stored as a labelled point list, any number of geometric heuristics may be applied to facilitate image analysis in a wide range of diagnostic situations and clinical environments.

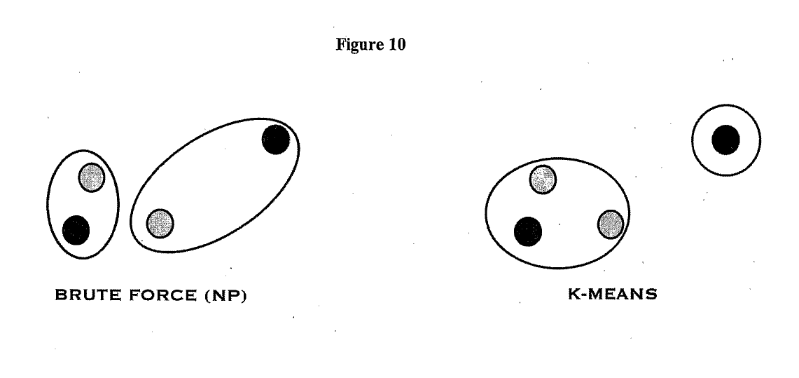

It should be noted that the simple edge-length comparison of all points in the image contemplated by the present invention outperformed other clustering methods such as k-means (Pattern Classification 2^{ed}, Duda, R. O. et al., New York, Wiley Interscience (2001)) for this domain. While k-means performed satisfactorily in making local clusters, it was unable to pair certain break-apart probes in their translocation configuration. This can be seen conceptually in FIG. 10. Since there are a limited number of probes present in any cell (usually less than ten), it is computationally tractable to do a brute-force comparison of their locations. This method would rapidly become computationally demanding if applied to a whole population of cells instead of a single cell. However, given the current architecture the computational vision system of the present invention will never have to deal with more than one cell at a time.

The present invention contemplates that individually tailored channel inhibition levels (e.g. noise rejection thresholds for histogram cropping), including but not limited to those disclosed herein, are essential to the detection of accurate probe groups. It is contemplated that such individual channel tailoring may be itself automated through use of a known standard, or through other means known in the art. Because of cellular auto-fluorescence in the frequency range near the green probe excitation wavelength, the green channel has a much higher overall background than the red channel for the studied FISH images. As such, a higher inhibition cropping level of 190 out of 255 was needed to filter out background artefacts. However, the red channel typically had much lower background levels and as a result only inhibition levels between 110 and 120 allowed successful probe detection in the most difficult cases. The same analysis considerations apply to any other channels that may be included in an analysis dealing with more than two colors, for example but not limited to, multiple probe staining coupled with staining of the cell nucleus with the dye DAPI (4′6-diamidino-2-phenylindole). As background levels may vary depending on the specific probes used and their relation to cellular material, inhibition level is an important input variable that depends on determining the preliminary domain knowledge.

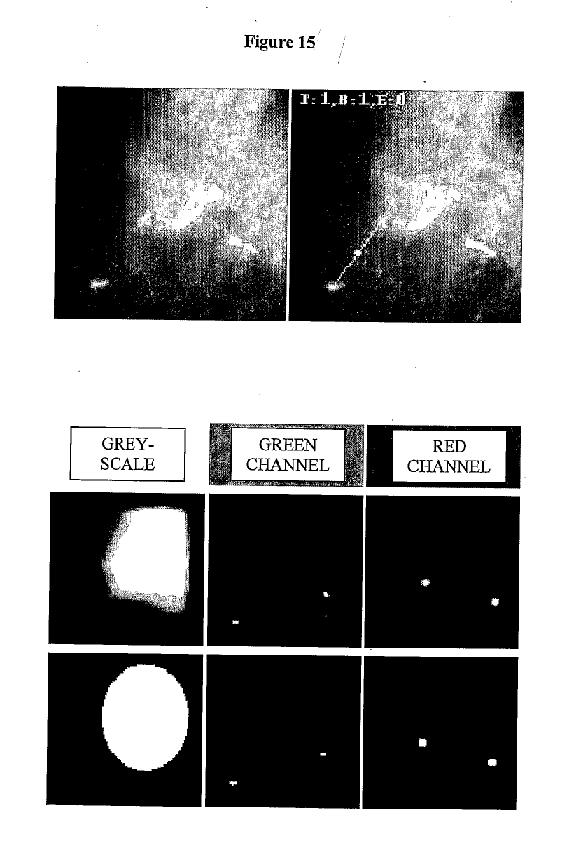

With regard to the boundary approximation: it was found that it was impossible to identify the correct probes in damaged or irregularly shaped cell nuclei (with high background intensity), such as those shown in FIG. 15, without the boundary approximation. For sharp, clear, cell images the cropping and boundary effects were negligible due to fact that the image background was already close to zero intensity—no erroneous edges were formed. However, for images with higher background level the approximation was essential.

It should also be noted that a dramatic reduction in processing time can be achieved during optical processing by sparsely sampling the area surrounding a DOG center point, and if the area is determined to be homogeneous (e.g. a uniform black image background) not performing DOG processing at that point. Depending on the complexity and inherent object spacing in a sample image, this heuristic could lead to a processing time reduction of several magnitudes (e.g. for an image with a widely spaced cells placed on a large black background, such as shown in FIG. 4). As shown in FIG. 4, the target box and image were produced by commercial image acquisition system (BioView Duet scanning unit, Rehovot, Israel).

Finally, it should be noted that damaged or irregularly shaped cell nuclei cannot be effectively modeled as ellipses, as seen in FIG. 15. This is an effective tool to rule out damaged cells from the viable sample population. By comparing the extracted cell ellipse aspect ratio and size to a viable sample standard it is possible to rule out additional noise artefacts and detected cells that were not useful for medical analysis. This holds with clinical practice, where incomplete and/or damaged cells are not considered during patient screening.

In a preferred embodiment, the computer vision system of the present invention system
(1) scans at low resolution for cell-like objects (using a selected color channel which stains predominantly and generally cells). It then makes a list of where each cell is in the image, noting the dimensions of each cell thereby determining the salient areas for analysis.
(2) Next, the system steps through the list of salient objects and retrieves just the portion of the image containing the cell, and then performs optical processing on it to extract red and green salient features (i.e. probes).
(3) The system then moves to next cell in the list, once again performing optical processing and continues until every whole cell has been analyzed for probe content.
(4) Once finished, it uses the extracted probe information to determine the relationship of probes in each cell and these results are tallied to generate a set of population statistics that may be used to determine medical information.

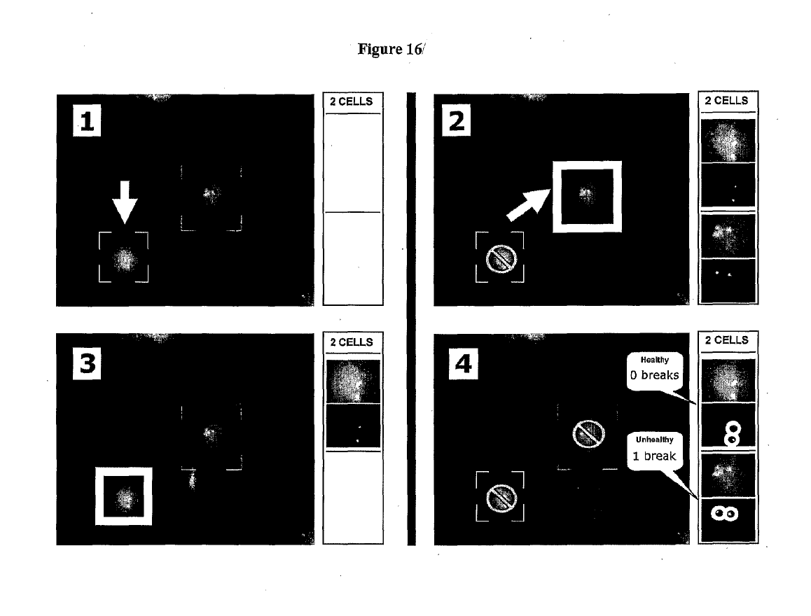

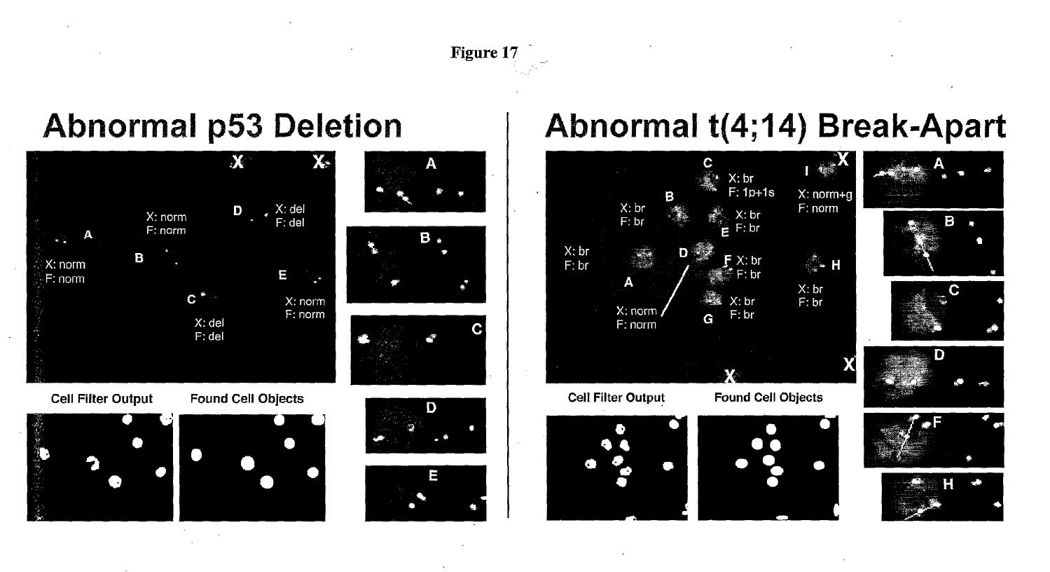

FIG. 16 shows the stepwise progression of the preferred embodiment. FIG. 17 shows the result of this embodiment as applied to a population image of cells. It can be seen in FIG. 17 that the predictions of the FISH expert (labelled X) are in concordance with the output of the preferred embodiment (labelled F). With minor adjustments, the invention can be readily applied to detecting cells stained with any kind of colorimetric marker, including but not limited to a wide variety of cellular phenotypes, functional properties or receptors.

### EXAMPLES

### General Materials & Methods

Reagents were purchased from Invitrogen (Carlsbad, Calif., USA) and all dilutions were performed with autoclaved Milli-Q water unless otherwise noted. Two buffers, 1×PBS (phosphate buffered saline) and 2×SSC (sodium citrate, sodium chloride, dilute from 20×SSC stock), were used in these FISH experiments (Beatty, B. et al. Oxford University Press. p. 255 (2002); Introduction to Fluorescence In Situ Hybridization: Principles and Clinical Applications, Andreeff, M., New York: Wiley-Liss p. 455 (1999)). The enzyme solution employed for digestion was made by mixing 1 ul of 25 mg/ml proteinase K stock to 1 ml of 2×SSC. Stock 95% ethanol was diluted to yield 70% and 85% ethanol solutions necessary for a series of dehydration and fixation steps; see procedure below. Seven FISH probes from Vysis (Downers Grove, Ill., USA) were used to detect various chromosomal abnormalities associated with MM. Four of the FISH probes targeted for the immunoglobulin heavy chain (IGH) locus associated with 14q32 translocations were used; namely, the LSI® IGH/FGFR3 Dual Color, Dual Fusion Translocation Probe, LSI® IGH/CCND1-XT dual color, dual fusion translocation probe, LSI® IGH/MAF dual color, dual fusion probe and the LSI® IGHC/IGHV Dual Color, Break Apart Probe. The three other probes are: D13S319 to detect deletions of chromosome 13, a mixture of CEP 17 and LSI® p53 to detect deletion of p53 and a CEP 1 to act as a control for a homemade probe directed to 1q21 locus.

Three homemade probes were prepared, each one composed by a control and a specific locus targeted probe and were directed to locus 1q21, 5q33.2-5qter and 19q13.4. To prepare these probes four BAC clones were purchased from Roswell Park Cancer Institute: RPCI11 89N18, RPCI11 307C12, RPCI11 91G17 and RPCI11 60A21; CTC 470E3 was also acquired. The clones were grown, DNA extracted and labelled either orange or green using a nick translation kit and fluorophores purchased from Vysis. These probes were tested on normal metaphases and against commercial controls directed to the same chromosomes The commercial probes were prepared as per the instructions provided by the vendor. NP-40, Nonidet P-40, a non-ionic detergent, was used in the post-hybridization wash solution and for optimum probe detection. Vectashield H-1000 anti-fading solution was used to reduce photobleaching during fluorescence imaging and was purchased from Vector Labs Inc. (Burlingame, Calif., USA). Ross rubber cement (Toronto, ON, Canada) was used to prevent evaporation during hybridization by covering wells on a microchip and by sealing coverslips on a microscope slide. As well, probes from other companies, for example Cytocell, have been tested using the invention and verified to work with on-chip FISH.

The three cell lines used for experiments were: RAJI (Burkitt's lymphoma), KMS12-BM (bone marrow, MM), and KMS18 (MM). Cell lines were cultured in RPMI 1640+10% Fetal Bovine Serum (FBS)+2 mM L-Glutamine+100 mM Hepes+0.25 mg/ml gentamicin and maintained at 0.5-2 million cells/ml; 5% CO² at 37° C. In addition, ex-vivo PBMC from three MM patients were purified as previously described (Pilarski, L. M. et al., Clin. Cancer Res., 6:585-596 (2000); Bergsagel, P. L. et al., Blood, 85:436-447 (1995)) and used for microchip-based FISH.

The microchip-based FISH protocol discussed below was performed on a custom designed microfluidic device fabricated in the University of Alberta Nanofabrication Facility. All microfluidic devices discussed in this paper were fabricated following standard glass etch and bonding protocols, as known in the art (Harrison, D. J. et al., Science, 261:895-897 (1993)). The microfluidic network that implements the microchip-based FISH protocol is illustrated in FIG. 7. FIG. 7(b) shows a sample cross-section of a microchannel in the microchip array, while FIG. 7(c) shows the combined mask layouts and dimensions of circulating microchip, which was employed for recirculating probes over immobilized cells. The gray lines are the features etched in the control layer for the pneumatic valves 701, 702, 703, 704, 705, while the black features are the fluidic network (eg. 707, 708, 709). As shown in cross section in FIG. 7(d)(e), the two glass substrates each adjacent to a thin PDMS membrane 714; this creates a movable diaphragm. FIG. 7(d) shows a cross section of a valve in closed position as pressure is applied to the control layer 716, thereby sealing the discontinuous fluid layer 717. FIG. 7(e) shows a sample cross section of a valve in open position with vacuum applied on control layer 716 creating a fluid pathway on fluidic layer 717.

The microchip in one embodiment shown in FIG. 7(a) consists of 10 straight channels 712 (nominal dimensions are 55 μm×310 μm×50 mm) and 20 access ports/wells 710, 711 (each containing ca. 1.5 μL). Channels 712 are etched in 0.5 mm 0211 Corning glass (Precision Glass and Optics, Santa Ana, Calif.) with access ports/wells 710, 711 also in the 0.5 mm substrate. 0.17 mm thick 0211 Corning glass cover plates were necessary to create a minimum working distance for high resolution imaging. The thickness of the cover plate can be varied as needed.

The Microfluidic Tool Kit, referred to as the μTK, was purchased from Micralyne (Edmonton, AB, Canada) and provided electrophoretic control of reagents and DNA samples for the straight channel microchip array. However the use of any of a variety of devices designed to run microfluidic chips, including portable or handheld instruments, is contemplated. The programmable application of high voltages to the microchip is fully controlled by the μTK via a compiled LabVIEW interface supplied by Micralyne (Sieben, V. J. et al., Electrophoresis, 26:4729-4742 (2005)). A custom plexi-glass enclosure was built that mounted onto a thermocycler and provided voltages to the microchip while temperatures were being applied. Another custom desktop system was designed and implemented that permitted temperature control and automated pneumatic valve actuation for the circulating microchip. Additionally, a software package was designed to provide automated and programmable control of valves and temperature, permitting the pumping and valving to be repeatedly applied without human intervention. It is contemplated that any of a number of types of pumping and valving strategies.

Fluorescence microscopy was performed on a Carl Zeiss Axioplan 2 microscope (Oberkochen, Germany) with the appropriate filter sets. The objective used for imaging was a Carl Zeiss water immersion lens, the Achroplan 63×W IR, with a numerical aperture of 0.9 and a working distance of 2.2 mm. This objective was necessary to resolve probes at a high magnification as it provided a sufficient working distance when imaging cells on the circulating microchip (layer of glass=1.1 mm). Images were captured with Metamorph (v. 7, Molecular devices, Downingtown, Pa., U.S.A.) and a Photometrics Cool Snap HQ charge-coupled device camera, 1392×1040 pixels (Roper Scientific, Trenton, N.J.). Imaging can be performed using a variety of devices, all within the scope of this invention, including those that create digital images of the stained cells within the channels for further computer vision analysis.

### Example 1

### Cell Preparation and Immobilization

Before FISH could be implemented within a microfluidic platform, it was necessary to develop a method for immobilizing cells within channels. From an initial concentration of ˜10 million cells per millilitre, ˜15,000 cells suspended in 1×PBS were added to each sample access port/well (1.5 μL). Capillary force moved the cells (in solution) into the microchannel 712, coating the surfaces over entire length the channel. Any remaining cells in the wells were removed, with a pipette. The microchips were then heated to an optimal temperature of 85° C. for 10 minutes. The remaining cell solution in the channels were removed with a filtered vacuum line (20 in·Hg) applied to port/well 711, or optionally and in the alternative 710. After temperature immobilization, approximately 70% of the cells remained adhered to the channel walls, with almost 90% of the cells immobilized on the bottom of the channel (etched surface). The temperature-induced immobilization procedure was employed for all microchip experiments. The number of cells used for immobilization can be adjusted to meet test requirements.

Following immobilization, the cells were enzymatically digested with proteinase K to facilitate entry of the DNA probes to enter the cell. The proteinase K was delivered to the cells by pipetting 1.5 μL of a diluted solution into the access port/well 710 and allowing capillary forces to fill the entire channel 712. The proteinase K solution was allowed to digest cells for 10 minutes and then removed by applying vacuum (20 in·Hg) to the channels 712 using access ports/wells 710, or optionally and in the alternative 711. The cells were washed with a continuous flow of 30 μL of 1×PBS through each channel 712 to ensure enzyme removal. Next, dehydration and fixation of the chromosomal DNA was performed by a series of ethanol treatments. 70%, 85% and then 95% ethanol solution was loaded into the channels and left for 1 or 2 minutes. Following the removal of the last ethanol treatment, vacuum was applied for 2 minutes to dry the cells.

### Example 2

### Circulating Microchip

A circulating microchip was designed such that the probe could be recirculated over the immobilized cells, facilitating more rapid and efficient hybridization. This required a microfluidic network and a pneumatic pumping and valving system, as illustrated by the combined mask layouts shown in FIG. 7(c). The circulating chip is built with three layers: a rigid layer 715 with fluid channels, a flexible middle layer 714 that acts as a controllable membrane, and an adjacent layer 713 with control channels and chambers for actuating the valves and pumps. Layer 715 consists of two discontinuous circular fluid channels 707 (nominal dimensions are 40 μm×580 μm with a radius of 5 mm) each with two wells 708, 709 (each containing ca. 1.5 μL) in 1.1 mm thick borofloat glass (Precision Glass and Optics, Santa Ana, Calif.). Middle layer 714 was a 0.254 mm thin sheet of PDMS (HT-6135, Bisco Silicons, Elk Grove, Ill., USA). Layer 713 was fabricated on 1.1 mm borofloat glass and had ten access ports 706 drilled to provide individual control over each valving chamber 701, 702, 703, 704, 705. This allowed either pressure (15 psi) or vacuum (20 in·Hg) to be applied to a valve chambers 701, 702, 703, 704, 705, thereby closing or opening the valves respectively.

Miniaturized valves 701, 702, 703, 704, 705 were used for active mixing during the hybridization phase of FISH. In traditional active mixing setups, a substantial volume of solution is contained off-chip, in the tubing and in the off-chip valves (effectively dead volume). When using relatively expensive reagents, such as the FISH probes, this conventional type of active mixing setup is uneconomical. Furthermore, it is difficult to maintain the off-chip solution at a uniform temperature. By integrating miniaturized valves on-chip the amount of expensive reagent used was minimized and uniform control of the temperature of the solution for denaturation and active mixing was maintained during the hybridization process. It was contemplated that several circulating channels can be chained in parallel with the same control lines for an increased level of automation.

### Example 3

### Hybridization within the Microchip

Using the chip design of FIG. 7(a), a probe solution described herein was added to the sample wells 710 of the chip and vacuum applied to the opposite well 711 to pull the viscous probe solution into the channel 712. The total volume of probe used was 1 μL (approximately 1/10th that used on conventional microscope slides). The wells 710, 711 were then blocked with rubber cement to prevent evaporation. Next, a set of thermal sequences permitted controlled denaturation of the chromosomal and probe DNA. The program sequence was as follows: a) 37° C. for five minutes; b) 75° C. for five minutes; c) hold at 37° C. For diffusion-based experiments, the probe was left in the channel to hybridize for the time duration desired, which ranged from 1-14 hours. Following hybridization the channel seals over wells/ports 710, 711 were removed and 20 μL of 0.4×SSC at 70° C. was flushed through the channels 712. The channels were then emptied, and filled with 2×SSC/0.01% NP-40 for one minute. These post-hybridization treatments ensured the removal of any cross hybridization (non-specific binding). Next, the cells were washed with 30 μL of 1×PBS in a continuous flow. Finally, channels 712 were filled with the anti-fading solution and imaging of the cells was completed with the fluorescence microscope indicated above.

### Example 4

### Hybridization with Electrokinetic Transport

Another set of microchip arrays using the chip design of FIG. 7(a), were also used for electrokinetic experiments and the chips were prepared as indicated above. However, after the thermocycling and before the hybridization, the rubber cement was removed, hybridization solution (purchased from Vysis) was added to fill the wells/ports 710, 711, and electrodes were lowered into the wells 710, 711. An electric field sequence was applied to gradually move the DNA probes down the channel 712 as follows: a) an electric field (10 V/cm) was applied for two minutes in one direction; b) one minute pause; c) the polarity was reversed and the field was applied for one minute; d) one minute pause. The electric field program was continuously repeated in this manner for one and four hours. An electric field strength of 10 V/cm was chosen to ensure that the environment close to the cell was not significantly disturbed and to minimize the current draw. Following the electrokinetic cycling, the same post-hybridization washes as described in Example 3 were applied to remove cross hybridization and any remaining probe.

### Example 5

### Hybridization with a Circulating Microchip

Using the chip design of FIG. 7(c), the chip was prepared as described in Example 2, with all valves 701, 702, 703, 704, 705 opened to allow the solutions to be passed through channel 707. The probe solution was then added to the sample well 708 of the chip and vacuum applied to well/port 709 pull the viscous probe solution into channel 707. With the channels now full, the two outer sealing valves 704, 705 were closed to prevent evaporation. Next, the thermal sequence described in Example 3 was applied. Following thermal sequencing, the temperature was set to 37° C., and a pump-based hybridization was performed. Peristaltic pumping was achieved by sequential opening and closing of valves 701, 702, 703. One pump cycle was completed every minute. The pumping program was repeated in this manner for one and four hours. After hybridization, all valves 701, 702, 703, 704, 705 were opened and the post-hybridization washes and procedures were completed.

### Example 6

### Image Analysis

To validate the efficacy of microchip-based FISH, an objective method was needed to compare the amount of cell background fluorescence to the detected probe intensity in imaged cells. As such, a signal-to-noise ratio was algorithmically computed from microchip and slide-based FISH images to measure hybridization efficiency. It was found that this signal-to-noise ratio was invariant to differing levels of image amplification, and proved to be an unbiased method to compare hybridization across all physical platforms.

Each cell image was manually cropped for individual analysis from the full image, with a buffer of at least twenty ambient background pixels between the observable cell edge and the image boundary. Cell images are typically comprised of three regions (as discernible by eye). Ambient image pixels (I_{α}) were defined as all contiguous pixels within thirty intensity levels (on an 8-bit greyscale) of an ambient reference pixel, i.e. the top left corner of each cropped cell region. Pixels in regions of specific probe hybridization (Iₚ) were defined as all those contiguous pixels within ten intensity levels of a manually selected reference pixel within the region. The cell background intensity level (I_{b}) was defined as the average intensity over all image pixels not identified as belonging to a probe or the ambient image background. Signal-to-noise values were computed as avg(Iₚ)/avg(I_{b})—the average intensity level of all probes within a cell (i.e the signal) divided by the background level for that cell (i.e the noise).

Although this process depends upon manual cropping and the selection of reference pixels, the use of all contiguous pixels minimizes the effects of user selection. The remainder of the processing is entirely automatic. The automated image-processing method can identify cell/probe boundaries. Signal-to-noise evaluation was performed on 10 to 30 red and green channel sample images for each hybridization condition (one, two, four, and fourteen hours), and the average value of all samples (with standard deviations) at each hybridization time was taken as the signal-to-noise ratio for that timepoint. Although this is a relatively small data set, the distribution appeared randomly distributed about the mean. This algorithm has been validated against human interpretation and provides an unbiased method of establishing the signal and noise levels.

When analyzing FISH results by either method, a simple set of criteria was applied. Considering the regions of specific hybridization to be circles, when using a break-apart probe set, the circles had to be more than two diameters apart to be considered translocated. The cells having all of their red-green circle pairs closer than two signal diameters apart were classified as normal.

### Example 7

### Use of the Computational Vision System of the Present Invention

Patient samples were processed using a commercial FISH imaging system on standard microscope slides, with the sample cells used in the present experiments identified by the commercial system as the best cell examples in a given population. This is the current image quality standard, and the images can be seen to have low background level, few noise artefacts, high contrast and bright localized probe objects. A second set of samples were processed using an alternate FISH preparation substrate. Both of these sample populations were used to test the portability and ability to detect probes in a variety of challenging conditions of the computational vision system of the present invention. In addition to the intact cells, several damaged or bad cells were presented to the system to test its ability to analyze cells with non-standard probe orientations, sharp background features, and nebulous cell areas. The system was additionally tested on two larger image samples containing a number of cells, as shown in FIG. 17.

The two libraries of sample cells were tested with a variety of ganglion receptive field sizes and channel inhibition levels. In addition, several forms of cropping were tested, to simulate the effects of a higher level algorithm singling out individual cell regions for analysis. As such, cells were tested with their natural extra-cellular background intensity, a completely black (absent) background intensity, and the selective subtraction of other cells from the local background region. It is important to note that a single set of inhibition and receptive field size parameters was used for all of the following results. These images were tested with the inhibition values (Grey=190/255, Green=190/255, Red: 110/255) and a receptive field size radius of (Type 1=10, Type 2=20). All images were 150 to 200 pixels on a side, with probes signals ranging in size from six to eight pixels in diameter. A broken cluster was defined as a pair with a distance of more than 24 pixels between probe centers. This parameter set was generated experimentally on one of the most difficult samples (AML01) and applied to the entire sample set. FIGS. 11-15 demonstrate the predictive ability of the present computational vision system with regard to a diverse range of sample image contrasts, feature clarities, and background levels. FIGS. 16 and 17 show the present computational vision system's ability to analyze larger more complex images in an automated fashion (i.e. extract cell and probe information and make relational assessments on these extracted objects with no human intervention and no prior training on a given test image) and obtain results comparable to those of a human FISH expert.

While particular embodiments of the present invention have been described in the foregoing, it is to be understood that other embodiments are possible within the scope of the invention and are intended to be included herein. It will be clear to any person skilled in the art that modification of and adjustments to this invention, not shown, are possible without departing from the spirit of the invention as demonstrated through the exemplary embodiments. The invention is therefore to be considered limited solely by the scope of the appended claims.

What is claimed is:

1. An automated computer vision system capable of assessing the presence, absence and location of a luminescent probe within a cell or population of cells comprising a computer readable memory a computer and an optical imaging device all in digital communication with each other wherein the optical imaging device is capable of receiving an optical image of a population of cells of interest and converting said optical image into a digital representation and wherein
a) Said optical imaging device transmits said digital representation to said computer readable memory;
b) Said computer scans said digital representation at low-resolution for cell-like objects;
c) Said computer creates a listing, capable of being referenced by the computer at some later time, of each cell-like object in the digital representation thereby generating a list of salient areas;
d) The computer chooses a salient area from the first element in said list of salient areas;
e) The computer retrieves the portion of said digital representation which contains at least said salient area for analysis and performs digital processing on said portion of the digital representation so as to identify, locate and store within said computer readable memory the location of at least one probe present in said portion of the digital representation;
f) The computer then chooses the next element in said list of salient areas, performing step e) above;
g) Step f) is repeated until a sufficient number of salient areas have been analysed, said sufficient number determined at the option of the computer system or by intervention of a human operator;
h) The system uses the location or at least one probe in each of the analyzed salient areas to determine the relationship of probes within each salient area; and
i) Said relationship of probes are used to generate a set of population statistics that may be used for analyzing the condition of said cells of interest.

2. A method of immobilizing cells in a microfluidic channel and preparing said cells for use in cellular analysis comprising
Taking a population of cells of interest, suspended in a fluid;
Filling a microfluidic channel with said population of cells of interest suspended in a fluid; and
Raising the temperature of said microfluidic channel to 55-95° C. for a period of time sufficient to allow immobilization of a portion of said population of cells of interest to said microfluidic channel;
Wherein time sufficient to allow immobilization of a portion of said population of cells of interest is determined by intervention of a human operator as the immobilization of a certain portion of cells of interest, either in terms of net number of cells immobilized, or alternatively as a percentage of total cells present in said population of cells of interest.

3. The method of claim 2 wherein the cellular analysis is FISH.

4. The method of claim 2 wherein the fluid is a buffer suitable for maintaining the size and shape of the individual cells making up the population of cells of interest.

5. The method of claim 4 wherein the buffer is 1× Phosphate Buffered Saline (PBS).

6. The method of claim 4 wherein the temperature is raised to 75-85° C.

7. The method of claim 6 wherein the temperature is raised to 75-85° C. for a period of 10 minutes.

8. A method of increasing the portion of cells of interest immobilized within a microfluidic channel comprising
Having at least one region within said microfluidic channel with a course surface;
Taking a population of cells of interest, suspended in a fluid;
Filling a microfluidic channel with said population of cells of interest suspended in a fluid so as to allow a portion of said population of cells of interest to come into fluid contact with said course surface of said microfluidic channel;
Raising the temperature of said microfluidic channel to 55-95° C. for a period of time sufficient to allow immobilization of a portion of said population of cells of interest to said microfluidic channel;
Wherein time sufficient to allow immobilization of a portion of said population of cells of interest is determined by intervention of a human operator as the immobilization of a certain portion of cells of interest, either in terms of net number of cells immobilized, or alternatively as a percentage of total cells present in said population of cells of interest; and

9. An apparatus for performing cellular analysis comprising
A first access port/well;
A second access port/well; and
At least one microfluidic channel;
Wherein said first access port/well is in fluid communication with said second access port/well by means of said at least one microfluidic channel;
And wherein said at least one microfluidic channel is of dimensions no greater than 110 μm×620 μm×100 mm.

10. The apparatus of claim 9 wherein the dimensions of the microfluidic channels are 55 μm×310 μm×50 mm.

11. The apparatus of claim 10 wherein the first access port/well and second access port/well each have a volume of 1.5 μL.

12. The apparatus of claim 10 wherein said microfluidic channels and said first and second access ports/wells are formed by etching a planar glass surface.

13. The apparatus of claim 10 wherein said microfluidic channels and said first and second access ports/wells are formed by the interface between a planar glass surface and a moulded flexible, plastic.

14. The apparatus of claim 13, wherein said moulded, flexible plastic is PDMS.
